(19) United States
(12) Patent Application Publication
(10) Pub. No.: US 2023/0364102 A1
(43) Pub. Date: Nov. 16, 2023

(54) COMPOSITIONS AND METHODS FOR THE TREATMENT OF NEUROLOGICAL DISORDERS

(71) Applicant: Duke University, Durham, NC (US)

(72) Inventors: Laurie Andolina, Durham, NC (US); Claudia Gonzalez-Hunt, Durham, NC (US)

(21) Appl. No.: 18/044,981

(22) PCT Filed: Sep. 9, 2021
(86) PCT No.: PCT/US2021/049639

§ 371 Date: Mar. 10, 2023

Related U.S. Application Data
(60) Provisional application No. 63/076,505, filed on Sep. 10, 2020.

(51) Int. Cl. A61K 31/5377; A61K 31/4745; C12Q 1/6883; A61P 25/28; A61K 45/06

(52) U.S. Cl. CPC A61K 31/5377; A61K 31/4745; C12Q 1/6883; A61P 25/28; A61K 45/06; C12Q 2600/156

(57) ABSTRACT

The present disclosure describes, in part, compositions and methods for the treatment of neurological disorders using an ATM inhibitor. The methods are useful for the increased treatment of subjects that express a mutant Leucine Rich Repeat Kinase 2 (LRRK2) having kinase activity as compared to the wild type LRRK2. Exemplary LRRK2 mutations include G2019S, R1441C, R1441G, R1441H, Y1699C, or I2020T.

### PRIOR RELATED APPLICATION

This application claims the benefit of and priority to U.S. Provisional Application No. 63/076,505 filed on Sep. 10, 2020, which is hereby incorporated by reference in its entirety.

### FIELD

This disclosure describes compositions and methods for treating neurological disorders.

### BACKGROUND

Parkinson's disease (PD) is the most common neurodegenerative movement disorder, affecting over one million people in the US. PD is widely accepted as a multifactorial disease—with both genetic and environmental contributions. Mutations in leucine-rich repeat kinase 2 (LRRK2) are believed to be a common cause of inherited and idiopathic PD. Recently we found that LRRK2 activation may play a role in idiopathic PD. The molecular mechanisms associated with LRRK2 that lead to pathology and neurodegeneration in PD are complex and not completely understood. To date, treatments are only symptomatic; they do not alter the inexorable progression of the disease. Even with expert treatment, PD patients typically deteriorate over time and endure considerable motor and non-motor disability in the years after diagnosis. Since there are no disease modifying therapies for PD, creative new approaches and perspectives are needed to identify novel therapeutic targets. This is an urgent medical need because, until we do, disease-modifying therapies will likely remain beyond reach and symptomatic treatments such as dopamine replacement will continue to be the only arsenal against PD.

Mitochondrial dysfunction is one of the key mechanisms underlying the pathogenesis of both idiopathic and familial PD. Mitochondrial complex I activity is decreased in the brain and systemically in subjects with PD. Inhibitors of complex I, such as rotenone, when administered to rodents and non-human primates mimic many of the behavioral, pathological and clinical features of PD. Additionally, many of the genes that have been reported to cause PD, including LRRK2, have been linked to mitochondrial pathways. The majority of LRRK2 is localized in the cytoplasm, with a fraction of LRRK2 associated with mitochondria. LRRK2 may exert its effect on mitochondrial function either directly or indirectly. The most studied LRRK2 mutation (G2019S) shows altered mitochondrial dynamics, trafficking, bioenergetics, and mitochondrial DNA damage in a variety of model systems. Although mitochondrial impairment is central to PD, the molecular mechanisms by which LRRK2 causes mitochondrial dysfunction are not fully elucidated.

DNA damage is often associated with oxidative stress and mitochondrial impairment, both of which are implicated in the pathogenesis of PD. DNA mutations and DNA damage are fundamentally different. DNA damage is a physical abnormality in the DNA such as oxidized bases or single- and double-strand breaks. Enzymes recognize and repair DNA damage to prevent downstream consequences such as cell death. In contrast, DNA mutations are changes in the base sequence of the DNA. Traditional sequencing technology will detect mutations, but not DNA damage. Mitochondrial DNA (mtDNA) damage that is unable to be repaired or eliminated by other mechanisms such as mitophagy, tends to accumulate and persist, rather than convert to a mutation. Notably, mtDNA is more susceptible to oxidative damage than nuclear DNA. Yet the specific role of mtDNA damage in PD remains unknown.

### BRIEF SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Provided herein is a method of treating a neurological disorder comprising administering to the subject a therapeutically effective amount of an ATM inhibitor. In some embodiments, the subject expresses a LRRK2 variant protein, wherein the LRRK2 variant protein has increased kinase activity as compared to a wild type LRRK2 protein. In some embodiments, the LRRK2 variant protein comprises one or more amino acid substitutions relative to SEQ ID NO: 1. In some embodiments, the one or more amino acid substitutions comprise one or more of G2019S, R1441C, R1441G, R1441H, Y1699C, or I2020T. In some embodiments, the LRRK2 variant protein has an amino acid sequence that is at least 90% identical to SEQ ID NO: 1 and comprises one or more of G2019S, R1441C, R1441G, R1441H, Y1699C, or I2020T.

In some embodiments, the neurological disorder comprises a neurodegenerative disease, for example, any one of Alzheimer's disease, Mild Cognitive Impairment, Pick's disease, Parkinson's disease, Huntington's disease, or a prion-associated disease. In some embodiments, the ATM kinase inhibitor is any one of KU-55933, Dactolisib (BEZ235), KU-60019, JU-59403, AZ31, AZ32, AZD0156, AZD1390, VE-821, Wortmannin, Torin 2, CP-466722, Berzosertib (VE-822), and the like.

Also provided herein is a method of rescuing LRRK2 genome instability in a subject suffering from a neurological disorder, the method comprising administering to the subject a therapeutically effective amount of an agent that inhibits the activity of ATM such that LRRK2 genome stability is rescued. the LRRK2 variant protein comprises one or more amino acid substitutions relative to SEQ ID NO: 1. In some embodiments, the one or more amino acid substitutions comprise one or more of G2019S, R1441C, R1441G, R1441H, Y1699C, or I2020T.

Also provided herein is kit comprising: (a).primers for detecting a genomic sequence encoding a LRRK2 variant protein in a biological sample, wherein the LRRK2 variant protein has increased kinase activity relative to wild type LRRK2, and (b). instructions for identifying the genomic sequence as encoding the LRRK2 variant protein.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-1D show that PD-linked LRRK2 mutations caused mtDNA damage in accordance with one embodiment of the present disclosure. FIG. 1A shows that, in neural cells differentiated from iPSCs, mtDNA lesions were increased in LRRK2 G2019S (black bars, n=3) and R1441C mutation carriers (gray bars, n=5) compared to healthy subjects (white bars, n=3). Controls are C1, C2, C3. Clones in order from left to right: L1, L2, L3, L4a, L4b, L5a, L5b, and L5c. Data collected from each iPSC clone was from 3 independent differentiations. FIG. 1B shows genome editing of the LRRK2 G2019S (n=3) abrogated mtDNA damage to control levels (n=3). FIG. 1C shows that there are elevated levels of mtDNA damage in HEK293 LRRK2G2019S/G2019S (n=3) cells relative to isogenic controls (n=3). FIG. 1D shows that ventral midbrain collected from GKI mice aged 4-7 months (n=8) exhibited greater levels of mtDNA damage than wild-type littermates (n=4). *p<0.05. Student t-test. Data presented as mean±SEM.

FIGS. 2A-2B show that mtDNA damage increased in dopaminergic neurons in environmentally-induced PD model and human idiopathic PD post-mortem brain in accordance with one embodiment of the present disclosure. FIG. 2A shows that, 24 hours following a single injection of rotenone (an mtDNA damage-inducing agent), mtDNA damage was increased in the ventral midbrain (*p<0.001, Student's t-test). n=8 vehicle, n=9 rotenone-treated animals. FIG. 2B shows that, using an assay (as disclosed in U.S. Pat. No. 11,001,890) developed by the inventors for a specific form of DNA damage (abasic sites) in nigral dopamine neurons from PD patients accumulate mtDNA damage. Age-matched postmortem specimens from PD and control subjects were stained for tyrosine hydroxylase (blue), TOM20 (a mitochondrial marker; red) and reacted with aldehyde reactive probe (ARP, a marker of abasic sites; green). The majority of TH neurons from PD patients accumulated abasic sites (55.1±17.69%, n=5) compared to healthy controls (7.8±2.6%, n=5), which was statistically significant (*p<0.05).

FIGS. 3A-3D show that LRRK2 kinase inhibitor exposure reversed mtDNA damage in LRRK2 and idiopathic PD in accordance with one embodiment of the present disclosure. FIG. 3A shows primary midbrain neuronal cultures expressing LRRK2 G2019S induced mtDNA damage, in contrast to LRRK2 wild-type or kinase dead mutant relative to GFP. FIG. 3B shows that human LRRK2 G2019S or GFP expressing neuronal cultures were treated with GNE-7915 for 6 h, which restored mtDNA integrity. (*p<0.05 for data in a,b, determined by one-way ANOVA with a Tukey's posthoc comparison). Modified from (3). FIG. 3C shows that primary rat midbrain neurons pre-treated with GNE-7915 for 24 h was able to prevent rotenone induced mtDNA damage. (d) 24 h treatment with a LRRK2 kinase inhibitor (1 μM) is sufficient to reverse mtDNA damage in idiopathic PD patient derived cells. (*p<0.001 for data in c,d determined by one-way ANOVA with a Tukey's posthoc comparison). Data are presented as mean±SEM. Experiments were conducted with at least three biological replicates.

FIGS. 4A-4C show the cleavage rate measurements using multiplex DNA repair assays in accordance with one embodiment of the present disclosure. FIG. 4A shows the cleavage efficiency of 8-oxoguanine. In humans, it is primarily repaired by DNA glycosylase OGG1. FIG. 4B shows abasic sites in the mitochondria are decreased in the LRRK2 G2019S patient-derived lymphoblastoid cells (n=3) compared to healthy controls (n=3). FIG. 4C shows DNA samples that were treated with FPG. Higher levels of damaged purines recognized by this N-glycosylase was found in LRRK2 G2019S patient-derived (n=3) relative to control cells (n=3). Data are presented as mean±SEM. (*p<0.05, determined by one-way ANOVA with a Tukey's posthoc comparison in (a) or student's t-test in (b/c).

FIGS. 5A-5G show activation of ATM in LRRK2 and a rotenone-induced in vitro PD model in accordance with one embodiment of the present disclosure. FIG. 5A shows representative Western blot of LRRK2G2019S/G2019S knock-in HEK293 cells (n=3) relative to isogenic controls (n=3), FIG. 5C shows LRRK2 G2019S knock-in heterozygotes/homozygotes (n=4) compared to wild-type litter mates (n=5) from the ventral midbrain aged 6-7 months, and FIG. 5E shows HEK293 cells treated with 100 nM rotenone (n=3) for 24 h compared to vehicle (n=3) treated cells. FIGS. 5B, 5D, and 5F show quantification demonstrating that ATM pSer1981 (kinase active) is increased in in vitro and in vivo LRRK2 G2019S model systems and an idiopathic PD model. FIG. 5G shows a representative image of the full Western blot. (*p<0.01, determined by student t-test). Of note, the ATM pSer1987 is the mouse equivalent of the human ATM pSer1981.

FIG. 6 shows LRRK2 G2019S-induced γ-H2AX foci are reversed by LRRK2 kinase inhibition in accordance with one embodiment of the present disclosure. (human LRRK2G2019S/G2019S knock-in (KI) HEK293 cells were stained for γ-H2AX foci or Hoechst after being treated with a LRRK2 kinase inhibitor (LKI) or vehicle for 24 h. The results show that γ-H2AX foci were significantly increased in LRRK2 G2019S KI cells (n=3) compared to isogenic controls (n=3) and levels returned to isogenic control baseline following LKI. (**p<0.0001, (*p<0.05 determined by one-way ANOVA with a Tukey's posthoc comparison.

FIGS. 7A-7B show an activated ATM-Chk2-p53 signaling pathway in accordance with one embodiment of the present disclosure. Western blot quantification of LRRK2G2019S/G2019S KI cells (n=3) (FIG. 7A) relative to isogenic controls (n=3) (FIG. 7B) was performed. Quantification shows that pChk2 and p53 are increased in LRRK2 G2019S KI cells compared to isogenic controls (*p<0.05, student t-test).

FIGS. 8A-8B show LRRK2 G2019S-induced γ-H2AX foci and mtDNA damage was reversed by ATM kinase inhibition in accordance with one embodiment of the present disclosure. Human LRRK2G2019S/G2019S KI HEK293 cells were stained for γ-H2AX foci or Hoechst treated with an ATM kinase inhibitor or vehicle for 24 hours. FIG. 8A show that the amount of γ-H2AX in LRRK2 G2019S KI cells was significantly higher than the isogenic control. FIG. 8B shows that, after adding ATM kinase inhibitor, the amount of γ-H2AX in LRRK2 G2019S KI cells returned to the level that was substantially identical to that in the isogenic control, i.e., LRRK2 G2019S KI γ-H2AX foci were reversed. (**p<0.0001 and *p<0.05 determined by one-way ANOVA with a Tukey's posthoc comparison. Exposure to the selective ATM kinase inhibitor KU60019 (24 h, 1 μM) is also sufficient to reverse mtDNA damage in LRRK2 G2019S patient-derived cells (n=3). mtDNA copy number was similar among groups. (*p<0.05, determined by one-way ANOVA with a Tukey's posthoc comparison).

FIG. 9 is a schematic showing a working model for the role of LRRK2 in mitochondrial genome integrity in genetic and idiopathic PD in accordance with one embodiment of the present disclosure.

FIG. 10 shows that exposure of LRRK2 kinase inhibitors also restores mtDNA damage to basal levels in accordance with one embodiment of the present disclosure. Healthy control (n=3) or LRRK2 G2019S patient derived lymphoblastoid cells (n=3) were treated with RA283 (a LRRK2 kinase inhibitor provided by Sanofi pharmaceuticals) with doses ranging from 1-100 nM for 1.5 h. Data are presented as mean±SEM. (*p<0.001, determined by one-way ANOVA with a Tukey's posthoc comparison).

FIG. 11 shows that knockdown of Rab10 (a GTPase) mediates mtDNA damage levels in healthy cells in accordance with one embodiment of the present disclosure. The results show that the levels of mtDNA damage increased with Rab10 knockdown in HEK293 cells (n=3). (*p<0.05, determined by one-way ANOVA with a Tukey's posthoc comparison).

FIG. 12 shows that rapamycin (an autophagy inducer) exposure restores mtDNA damage back to healthy control levels in accordance with one embodiment of the present disclosure. DA neurons differentiated from iPSCs harboring the LRRK2 G2019S mutation (n=2) had increased mtDNA damage compared to healthy control (n=2). Treatment with rapamycin for 24 h restored mtDNA damage levels. Data are presented as mean±SEM.

FIG. 13 shows oxygen consumption rate (OCR), which was measured from a mitochondrial enriched fraction from rat whole brain in accordance with one embodiment of the present disclosure. Freshly isolated mitochondria from different brain regions, the degree of coupling between the electron transport chain and the oxidative phosphorylation machinery using different substrates and injections using the coupling assay will be performed.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used throughout, by “subject” is meant an individual. The term “subject” and “patient” are used interchangeably herein and refer to both human and nonhuman animals The subject can be an adult subject or a pediatric subject. Adult subjects include subjects older than eighteen years of age. Pediatric subjects include subjects ranging in age from birth to eighteen years of age. Preferably, the subject is an animal, for example, a mammal such as a primate, and, more preferably, a human. Non-human primates are subjects as well. The term subject includes cats, dogs, reptiles, amphibians, livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

As used herein, a “biological sample” includes any sample obtained from a subject. A sample may contain tissue, cells, proteins, nucleic acids or other cellular matter. A sample may also be the liquid phase of a body fluid from which sedimentary materials have been substantially removed. Exemplary samples include, but are not limited to, blood samples containing peripheral blood mononuclear cells (PBMCs), plasma, urine samples containing urinary cells, fluid “supernatant” that is substantially free of cells, a sample of bronchoalveolar lavage fluid, a sample of bile, pleural fluid or peritoneal fluid, or any other fluid secreted or excreted by a normally or abnormally functioning allograft, or any other fluid resulting from exudation or transudation through an allograft or in anatomic proximity to an allograft, or any fluid in fluid communication with the allograft. A sample may also be obtained from essentially any body fluid including: blood (including peripheral blood), lymphatic fluid, sweat, peritoneal fluid, pleural fluid, bronchoalveolar lavage fluid, pericardial fluid, gastrointestinal juice, bile, urine, feces, tissue fluid or swelling fluid, joint fluid, cerebrospinal fluid, or any other named or unnamed fluid gathered from the anatomic area in proximity to the allograft or gathered from a fluid conduit in fluid communication with the allograft. For example, the sample can be a urinary cell sample. A “post-transplantation sample” refers to a sample obtained from a subject after the transplantation has been performed.

As used throughout, the term “gene” refers to a nucleic acid, DNA or RNA, involved in producing or encoding a polypeptide. It may include non-coding regions preceding and following the coding region (leader and trailer) as well as intervening sequences (introns) between individual coding segments (exons). As used throughout, the term “nucleic acid” or “nucleotide” refers to deoxyribonucleic acids (DNA) or ribonucleic acids (RNA) and polymers thereof in either single- or double-stranded form. It is understood that when a DNA sequence is described, its corresponding RNA is also described, wherein thymidine is represented as uridine. Unless otherwise indicated, a particular nucleic acid sequence also implicitly encompasses modified variants thereof, alleles, orthologs, SNPs, and complementary sequences as well as the sequence explicitly indicated.

As used herein, the term polynucleotide or nucleic acid includes nucleotide polymers of any number. The term polynucleotide can, for example, have less than about 200 nucleotides. However, other polynucleotides can have more than 200 nucleotides. Probes and primers are polynucleotides. Primers can, for example, have between 5 and 100 nucleotides, or have about 15 to 100 nucleotides. Probes can have the same or longer lengths. For example, probes can have about 16 nucleotides to about 10,000 nucleotides. The exact length of a particular polynucleotide depends on many factors, which in turn depend on its ultimate function or use. Some factors affecting the length of a polynucleotide are, for example, the sequence of the polynucleotide, the assay conditions in terms of such variables as salt concentrations and temperatures used during the assay, and whether or not the polynucleotide is modified at the 5′ terminus to include additional bases for the purposes of modifying the mass: charge ratio of the polynucleotide, or providing a tag capture sequence which may be used to geographically separate a polynucleotide to a specific hybridization location on a DNA chip, for example.

The term “identity” or “substantial identity”, as used in the context of a polynucleotide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, about 20 to 50, about 20 to 100, about 50 to about 200 or about 100 to about 150, in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

As used herein, the term “substantially identical,” when referring to the relationship of two values, means that the difference between the two values is less than 40%, less than 30%, less than 25%, less than 20%, less than 10%, or less than 5% of the smaller value.

As used herein, the term “ATM Kinase Inhibitor(s)” refers to any compound, drug, agent and the like that is capable of decreasing and/or inhibiting the function of ataxia telangiectasia mutated (ATM), a core component of the DNA repair system. In some non-limiting embodiments, the present invention provides for a method of treating a neurodegenerative disease in a subject, comprising administering, to the subject, an amount of an ATM kinase inhibitor effective treating a neurological disorder and/or rescuing LRRK2 genomic instability in a subject suffering from a neurological disorder. A specific, non-limiting example of an ATM Kinase inhibitor which may be used according to the present disclosure include, but are not limited to, KU-55933, Dactolisib (BEZ235), KU-60019, JU-59403, AZ31, AZ32, AZD0156, AZD1390, VE-821, Wortmannin, Torin 2, CP-466722, Berzosertib (VE-822), and the like. In some embodiments, the ATM kinase inhibitor comprises AZD1390. In other embodiments, the ATM kinase inhibitor comprises KU60019.

As used herein, the terms “neurological diseases” or “neurological disorders” are used interchangeably and refer to a host of undesirable conditions affecting neurons in the brain of a subject. Representative examples of such conditions include, without limitation, Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders. As used herein, the term “movement disorder” includes neurological diseases or disorders that involve the motor and movement systems, resulting in a range of abnormalities that affect the speed, quality and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathesia, (x) muscle rigidity, defined herein as resistance of a muscle to stretch; (xi) postural instability; (xii) bradykinesia; (xiii) difficulty in initiating movements; (xiv) muscle cramps; (xv) dyskinesias; and (xvi) myoclonus.

As used herein, the term “neurodegenerative disease” or “neurodegenerative disorder” encompass a subset of neurological diseases/disorders characterized by involving a progressive loss of neurons or loss of neuronal function. Accordingly, the term “neurodegeneration” refers to the progressive loss or function of at least one neuron or neuronal cell. The ordinarily skilled artisan will appreciate that the term “progressive loss” can refer to cell death or cell apoptosis. The ordinarily skilled artisan would further appreciate that “neuronal cell loss” refers to the loss of neuronal cells. The loss of neuronal cells may be a result of a genetic predisposition, congenital dysfunction, apoptosis, ischemic event, immune-mediated, free-radical induced, mitochondrial dysfunction, lesion formation, misregulation or modulation of a central nervous system-specific pathway or activity, chemical induced, or any injury that results in a loss of neuronal cells, as well as a progressive loss of neuronal cells. Thus, a neurodegenerative disorder or neurodegenerative disease, as used in the current context, includes any abnormal physical or mental behavior or experience where the death of neuronal cells is involved in the etiology of the disorder, or is affected by the disorder. As used herein, neurodegenerative diseases encompass disorders affecting the central and peripheral nervous systems, and include such afflictions as memory loss, stroke, dementia, personality disorders, gradual, permanent or episodic loss of muscle control. Examples of neurological disorders or diseases for which the current invention can be used preferably include, but are not limited to, Alzheimer's Disease, Parkinson's Disease, Huntington's Disease, amyotrophic lateral sclerosis (ALS), Pick's disease, prion diseases, dystonia, dementia with Lewy bodies, multiple system atrophy, progressive supranuclear palsy, Friedreich's Ataxia, temporal lobe epilepsy, stroke, traumatic brain injury, mitochondrial encephalopathies, Guillain-Barre syndrome, multiple sclerosis, epilepsy, myasthenia gravis, chronic idiopathic demyelinating disease (CID), neuropathy, ataxia, dementia, chronic axonal neuropathy and stroke. In certain embodiments, the neurological disorder/neurodegenerative disease comprises Parkinson's disease.

Unless otherwise defined, all technical terms used herein have the same meaning as commonly understood by one of ordinary skill in the art to which this disclosure belongs.

The present disclosure provides, in part, compositions and methods for the treatment of a neurological disorder that comprises, consists of, or consists essentially of administration of an ATM kinase inhibitor.

### Methods of Diagnosis

One aspect of the present disclosure provides for methods of diagnosing a neuro-degenerative disorder, for example, a neurodegenerative associated with LRRK2 mutations and/or genome instability, comprising determining the presence of LRRK2 mutations and/or genomic instability in the subject.

The measurements may be derived from a biological sample taken from the subject. As used herein, the term “biological sample” includes, but is not limited to, a sample containing tissues, cells, and/or biological fluids isolated from a subject. Examples of biological samples include, but are not limited to, tissues, cells, biopsies, blood, lymph, CFS, serum, plasma, urine, saliva, mucus and tears. Alternatively, the biological sample may comprise extracellular fractions collected from cultured patient-derived cells (e.g., primary cells, immortalized cells, and/or iPSC lines). In some embodiments, the biological sample may be derived from samples taken from the periphery, e.g., peripheral blood samples. In a particular, non-limiting example, the method comprises determining the mutational status and/or genomic instability of LRRK2, wherein the presence of such mutation and/or genomic instability indicates that the subject suffers from a neurological disorder. The measurement of phosphoinositide metabolism may be achieved by any method known in the art.

### LRRK2

LRRK2 is a protein kinase that belongs to the LRRK/ROCO class of protein kinases. The amino acid sequence of wild type human LRRK2 is provided in SEQ ID NO: 1. It possesses an LRR (leucine-rich repeat) motif, a Ras-like small GTPase, a conserved C-terminal of Roc (COR) domain. The protein kinase domain of LRRK2 belongs to the tyrosine-like serine/threonine protein 1287), a small GTPase domain (residues 1335-1504), a C-terminal of Roc (COR) domain (residues 1517-1843), a serine/threonine protein kinase domain (residues 1875-2132) and a motif that has low resemblance to a WD40 repeat (residues 2231-2276).The majority of LRRK2 is localized in the cytoplasm, with a fraction of LRRK2 associated with mitochondria.

### LRRK2 Genome Instability and LRRK2 Mutations

As used herein, the term “LRRK2 genome instability” refers to an increased amount of mitochondria DNA damage in a subject as a result of the presence of one or more mutations in the LRRK2 protein as compared to the amount of the mitochondria DNA damage in a control subject (or population thereof). As used herein, the term “LRRK2 genome instability is rescued” refers to a reduction of the amount of mtDNA damage that result from the one or more LRRK2 mutations in the subject to a level that is substantially identical to the level in a control subject. In some embodiments, the control subject is a subject that expresses wild type LRRK2 protein. In some embodiments, the control subject is a healthy individual that does not have PD. In some embodiments, a subject having LRRK2 genome instability has a mtDNA lesion frequency that is at least 20%, at least 30%, at least 40%, at least 50%, at least 60%, at least 70%, at least 80%, and/or at least 90% higher than the mtDNA lesion frequency in a control subject.

As used herein, LRRK2 protein refers to both wild type LRRK2 and also LRRK2 variants (i.e. LRRK2 mutants), such as those disclosed in Wojewska, D. N. and Kortholt, A., Biomolecules 2021, 11(8), Article No. 1101, the entire disclosure of which is herein incorporated by reference for all purposes. In some embodiments, a LRRK2 variant has increased kinase activity as compared to the wild type LRRK, i.e., the one or more mutations in the LRRK2 variant are gain-of-function mutation. Non-limiting examples of a LRRK2 variant that has increased kinase activity include any of the G2019S variant (which comprises a single amino acid substitution G2019S as compared to SEQ ID NO: 1), the R1441C variant (which comprises a single amino acid substitution R1441C as compared to SEQ ID NO: 1), the R1441G variant (which comprises a single amino acid substitution R1441G as compared to SEQ ID NO: 1), the R1441H variant (which comprises a single amino acid substitution R1441H as compared to SEQ ID NO: 1), the Y1699C variant (which comprises a single amino acid substitution Y1699C as compared to SEQ ID NO: 1), and/or the I2020T variant (which comprises a single amino acid substitution I2020T as compared to SEQ ID NO: 1). In some instances, the LRRK2 variant has one or more of the following amino acid substitutions as compared to wild type LRRK2 protein (SEQ ID NO:1): G2019S, R1441C, R1441G, R1441H, Y1699C, and/or I2020T.

In some embodiments, the LRRK2 variants have at least 60%, 65%, 70%, 75%, 80%, 85%, 90%, 95%, and/or 99% identity to any one of SEQ ID NO: 1, provided that the LRRK2 variant has increased kinase activity as compared to the wild type LRRK2. In some embodiments, the LRRK2 variants has one, two, three, four, or five single amino acid mutations (substitutions, deletions, or insertions) relative to SEQ ID NO: 1, provided that the LRRK2 variant has increased kinase activity as compared to the wild type LRRK2.

As used herein, having increased kinase activity refers to that the kinase activity of the LRRK2 variant is at least 20%, 25%, 30%, 35%, 40%, 45%, 50%, 55%, 60%, 65%, 70%, 75%, 80%, 85%, 90%, 95%, 100%,and/or 200% and/or at least 1.5 fold, at least 2 fold, at least 5 fold, at least 10 fold, and/or at least 15 fold or higher than the kinase activity of the wild type LRRK2 protein as determined using the same assay. The kinase activity of the variant can be assessed by analyzing phosphorylation of a substrate of the kinase. Exemplary substrates of the LRRK2 include moesin, MBP, and LRRKtide as disclosed in Jaleel, et al., Biochem J. 2007 Jul. 15, 405(Pt 2): 307-317. Methods of analyzing LRRK2 kinase activity are well known and also disclosed, for example, in Jaleel, et al., Biochem J. 2007 Jul. 15, 405(Pt 2): 307-317. Mutations in LRRK2 can be detected using a variety of methods suitable for detecting mutations. Useful techniques include, without limitation, assays such as polymerase chain reaction (PCR) based analysis assays, sequence analysis assays, electrophoretic analysis assays, restriction length polymorphism analysis assays, hybridization analysis assays, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, and denaturing gradient gel electrophoresis, all of which can be used alone or in combination.

Any of a variety of different primers can be used to amplify an individual's nucleic acid by PCR in order to determine the presence or absence of a mutation in LRRK2 disclosure. As understood by one skilled in the art, primers for PCR analysis can be designed based on the sequence flanking the target sequence in the LRRK2 gene. As a non-limiting example, a primer can contain from about 15 to about 30 nucleotides of a sequence upstream or downstream of the the target sequence in the gene of interest. Such primers generally are designed to have sufficient guanine and cytosine content to attain a sufficiently high melting temperature to allow for a stable annealing step in the amplification reaction. Several computer programs, such as Primer Select, are available to aid in the design of PCR primers.

Sequence analysis can also be useful for determining the presence or absence of a particular variant or haplotype in the gene or locus of interest. As is known by those skilled in the art, a variant allele of interest can be detected by sequence analysis using the appropriate primers, which are designed based on the sequence flanking the polymorphic site of interest in the gene or locus of interest. For example, a variant allele in a gene or locus of interest can be detected by sequence analysis using primers designed by one of skill in the art. Additional or alternative sequence primers can contain from about 15 to about 30 nucleotides of a sequence that corresponds to a sequence about 40 to about 400 base pairs upstream or downstream of the polymorphic site of interest in the gene or locus of interest. Such primers are generally designed to have sufficient guanine and cytosine content to attain a high melting temperature which allows for a stable annealing step in the sequencing reaction.

### Detecting Mitochondria DNA Damage

In some embodiments, subjects who can benefit from the ATM inhibitor have increased mitochondria DNA (mtDNA) damage. In this disclosure, the term DNA damage refers to presence of DNA lesions (damaged sites) in cellular DNA, including lesions such as base alterations, base deletions, sugar alterations, and strand breaks). DNA lesions can be caused by depurination, depyrimidination, deamination of nucleotides, which can result from a variety of pH and temperature-dependent reactions. DNA can also be damaged by reactive oxidative species (ROS) that are generated by metabolic processes in the cell. Depending on what part of the DNA is reacted with, ROS can cause a range of lesions including strand breaks and removal of bases. In addition, ionizing radiation (e.g. X-rays) and ultraviolet radiation each can cause DNA lesions. DNA damage also includes chemical adducts (covalently attached groups) on DNA. Such adducts may come from a variety of sources, including lipid oxidation, cigarette smoke, and fungal toxins. These adducts attach to the DNA in different ways, so there are a variety of different effects from the adducts as well.

In contrast to conventional assays for mtDNA damage, which vary in sensitivity and have significant limitations, the method disclosed herein can be performed using a higher-throughput analysis in real time as described in U.S. Pat. No. 11,001,890, the entire content of which is herein incorporated by reference. In some embodiments, the method is a quantitative PCR (QPCR) based assay, which dispenses the step of isolating mitochondria typically required by traditional methods used in mtDNA analysis. This technique is based on the principle that various types of DNA damage have the propensity to slow down or block DNA polymerase progression. Thus, if equal amounts of DNA from different samples are amplified under identical conditions, the sample DNA with the least amount of DNA damage will amplify to a greater extent than DNA that is damaged. Therefore, the PCR-based assay disclosed herein can detect numerous types of DNA damage or DNA repair intermediates such as abasic sites and single- and double-strand breaks.

It is recognized that DNA content can vary in mitochondria from different cells or tissues. Therefore, in some embodiments, to normalize for mtDNA copy number, a small mitochondrial DNA fragment is amplified and the small mitochondrial PCR product so produced comprises no longer than 300 base pairs, no longer than 250 base pairs, no longer than 200 base pairs, or no longer than 150 base pairs. Amplification of this short fragment reflects only undamaged DNA due to the low probability of introducing damage in small genome segments. This small mitochondrial PCR product can be used as an internal control for mtNDA copy number and used to normalize the data obtained with the mitochondrial PCR product of interest (see, Furda et al., Methods. Mol. Biol. 2014; 1105: 419-37, doi: 10.1007/978-1-62703-739-6_31), which are typically larger than 300 base pairs, for example, larger than 400 base pairs, larger than 500 base pairs, or larger than 1000 base pairs.

Various DNA polymerases can be used in the QPCR assay for detection of mtDNA damage. In some embodiments, each reaction mixture comprises one or more DNA polymerases. In one embodiment, the one or more DNA polymerses include a Taq polymerase. In some embodiments, the one or more DNA polymerases include a DNA polymerase having proofreading activity. In one embodiment, the one or more DNA polymerases include KAPA LongRange Hot Start DNA polymerase. Exemplary methods for detection of mtDNA are disclosed in Sanders et al., Curr. Protoc. Toxcol. 2018 May; 76(1) e50, doi: 10.1002/cptx.50; and also Gonzalez-Hunt et al., Curr. Curr Protoc Toxicol 2016 Feb 1; 67:20.11.1-20.11.25, doi: 10.1002/0471140856.tx2011s67. The entire disclosure of said publication is herein incorporated by reference.

In some embodiments, the primers are designed to amplify a mitochondria fragment that is at least 2 kb, at least 3 kb, at least 5 kb, at least 6 kb, or at least 10 kb. In some embodiments, the primers are designed to amplify a mitochondria fragment corresponding to human beta globin region on chromosome 11. In some embodiments, the primers are 5′-TCT AAG CCT CCT TATTCG AGC CGA-3′(SEQ ID NO: 4) and 5′-TTT CAT CAT GCG GAGATG TTG GAT GG-3′ (SEQ ID NO: 5).

In some embodiments, the primers are used to amplify a 12.2 kb mtDNA fragment from region of the DNA polymerase gene beta, corresponding to GenBank Accession no. L11607. In some embodiments, the primers are: 5′-CAT GTC ACC ACT GGACTC TGC AC-3′(SEQ ID NO: 6) and 5′-CCT GGA GTA GGA ACA AAA ATT GCT G-3′ (SEQ ID NO: 7). A short mtDNA fragment (221 bp) can be used as internal control and amplified using primers such as, for example, 5′-CCC CAC AAA CCC CATTAC TAA ACC CA-3′(SEQ ID NO: 8) and 5′-TTT CAT CAT GCG GAGATG TTG GAT GG-3′(SEQ ID NO: 9).

In some embodiments, QPCR are performed and the reaction product is mixed with a nucleic acid stain (e.g., a fluorescent nucleic acid stain such as picogreen) and incubated for a period of time (e.g., at room temperature, in the dark) to allow development of the fluorescent signal.

Quantification of mtDNA Damage

In some embodiments, the amount of signal from the amplification product of the mtDNA of interest is normalized for copy number difference using the small mitochondrial PCR product (the internal control) described above. Various approaches can be used for the normalization to account for the copy number difference including, for example, those disclosed in U.S. Pat. No. 11,001,890. In one exemplary approach, to normalize, divide each sample's small mitochondrial amplification product signal value is divided by the average of all small mitochondrial amplification products to get a correction factor for each sample. Then, each sample's large mitochondrial value is divided by its correction factor. This is the normalized large mitochondrial fluorescence value. Next, the normalized fluorescence values of each sample is divided by the average normalized fluorescence value to get a ratio. The negative natural log (−ln) of the ratio provides the lesion frequency per fragment. This value is normalized to the number of lesions/10 kb. Examples of this calculation can be found in Gonzalez-Hunt et al., Curr. Protoc Toxicol. 2016; 67:20.11.1-20.11.25. In some embodiments, the amount of mtDNA can be represented by DNA lesion frequency, i.e., the number of DNA lesions per a fixed length of DNA. For example, a DNA lesion frequency can be expressed as the number of lesions/10 kb mtDNA.

In one embodiment, increased lesion frequency in mitochondrial DNA can reflected by a shift in the linear portion of a graph of the number of cycles versus fluorescence. See, e.g., U.S. Pat. No. 11,001,890. A decrease in fluorescence at a specific cycle within the linear range indicates that the subject has Parkinson's disease. Thus, the lesion frequency is compared to the lesion frequency in mitochondrial DNA in a control sample, such as a sample form a healthy subject that does not have the neurodegenerative disease, such as Parkinson's disease or Parkinsonism, or a standard value or curve.

In some embodiments, at least three biological samples are analyzed per condition, with at least three separate QPCR runs to calculate the average lesion frequency.

In some embodiments, the subject has mtDNA damage (e.g., associated with the presence of the LRRK2 mutations) characterized in a lesion frequency of 0.05 to 3 lesions/10 Kb mtDNA. In some embodiments, the subject has a mtDNA lesion frequency that is at least 20%, at least 30%, at least 40%, at least 50%, at least 60%, at least 70%, at least 80%, at least 90% higher than the mtDNA lesion frequency in a control subject. In some embodiments, the control subject is a subject that expresses a wild type LRRK2. In some embodiments, the control subject is a healthy individual that does not have PD.

The QPCR-based assay described above is extremely sensitive, allowing for the detection of very low quantities of damage that are biologically relevant; to detect numerous types of damage and therefore does not rely on assay off of a single chemical entity. Any region of DNA or gene of interest can be specifically amplified, based on primary selection, and therefore directly probed. The large mtDNA fragment that comprises most of the mitochondrial genome can be amplified. This method uses genomic DNA, which includes both nuclear DNA and mtDNA—there is no need to separately isolate mitochondria (or mtDNA). In some embodiments, the nuclear DNA and mtDNA are not separated prior to analysis. In some embodiments, the nuclear DNA and mtDNA are separated prior to analysis of either or both. The method can measure DNA damage in real time. The assay is a medium throughput, i.e., performed in a 96-well platform. It is more sensitive, efficient, and reliable compared to traditional approaches. As such, the overall time spent on detecting mtDNA is reduced.

### Methods of Treatment

Another aspect of the present disclosure provides a method of treating a neurological disorder comprising, consisting of, or consisting essentially of administering to the subject a therapeutically effective amount of an agent that inhibits the activity of ATM such that the neurological disorder is treated. In some embodiments, the subject has been determined to have LRRK2 genome instability. In some embodiments, the subject expresses an LRRK2 variant protein that has increased kinase activity. In some embodiments, the subject expresses an LRRK2 variant protein comprising one or more of substitution mutations G2019S, R1441C, R1441G, R1441H, Y1699C, and/or I2020T.

In another aspect, the present disclosure provides a method of rescuing LRRK2 genome instability in a subject suffering from a neurological disorder, the method comprising, consisting of, or consisting essentially of administering to the subject a therapeutically effective amount of an agent that inhibits the activity of ATM such that LRRK2 genome stability is rescued.

### ATM

ATM is a member of the PIKK family and participates in the DNA damage response. ATM function is critical for both nervous system and mitochondrial homeostasis. ATM is activated by DNA damage and phosphorylates several key proteins that initiate the DNA damage checkpoint, cell cycle arrest, DNA repair or apoptosis. Thus, excessive activation of the DNA damage response can lead to neurodegeneration. Autophosphorylation of ATM at position S1981, resulting in phosphorylated Ser1981 or pSer1981, is correlated with its DNA damage-mediated activation.

### Relationship Between LRRK2 and ATM

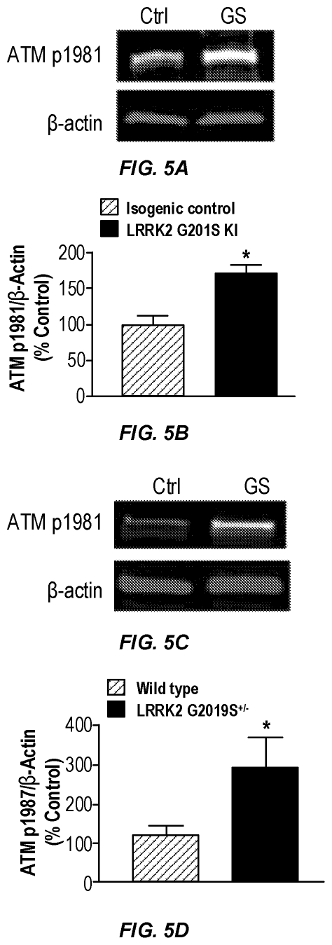

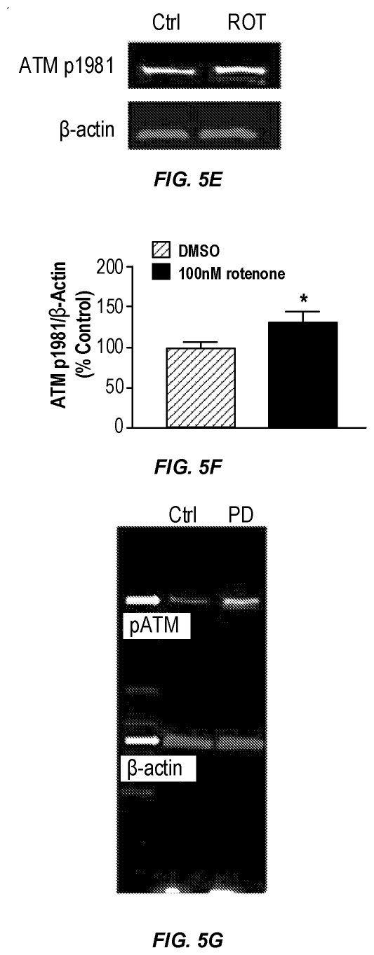

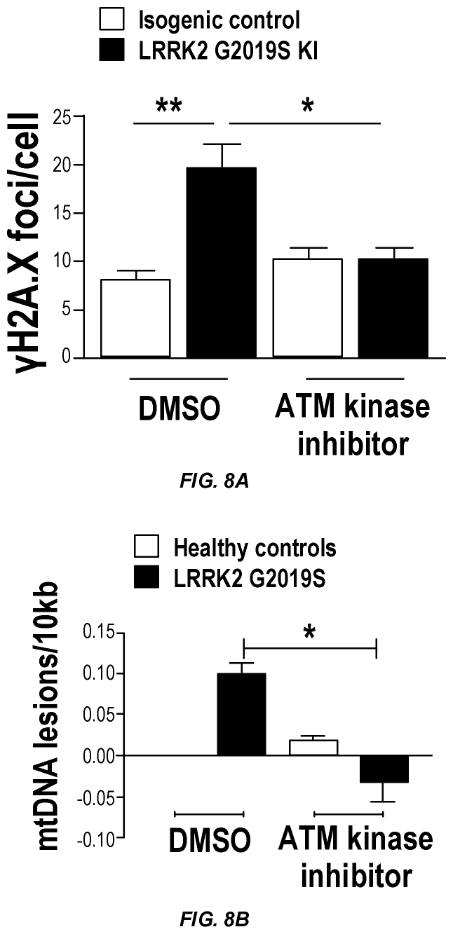

ATM is a serine/threonine kinase protein kinase that is recruited and activated by DNA double-strand breaks. It phosphorylates several key proteins that initiate activation of the DNA damage checkpoint, leading to cell cycle arrest, DNA repair or apoptosis. Several of these targets, including p53, CHK2, BRCA1, NBS1 and H2AX are tumor suppressors. As described in this disclosure, certain gain-of-function LRRK2 mutants have been determined to be associated with higher levels of activated ATM. First, ATM pSer1981 was found in LRRK2 G2019S cells by a quantitative phosphoproteomics screen. Second, levels of ATM pSer1981 were higher in both an in vitro model (LRRK2G2019S/G2019S KI HEK293 cells) and in vivo LRRK2 G2019S model (ventral midbrain derived from LRRK2 GKI mice). See FIGS. 5A-5D. Third, immunoflurorescence confocal microscopy analysis demonstrated that active ATM (i.e., ATM pSer1769) is detected in human LRRK2 G2019KI cells but not in isogenic controls. See also the priority application U.S. Provisional 63/076,505, FIG. 8. Imaging analysis showed that LRRK2 G2019S was associated with an increase in ATM pSer1981 levels (i.e., the activated ATM) in the cell nucleus. Activated ATM staining was predominantly in the nucleus and cytoplasmic expression showed no appreciable overlap with the mitochondrial marker. See U.S. Provisional Application No. 63/076,505, FIG. 16. These results support that there is a functional relationship between LRRK2 and ATM. Exposure to rotenone, an mtDNA damage-inducing agent, also increased ATM pSer1981 levels (FIGS. 5E, 5F).

### ATM Kinase Inhibitors

In some embodiments, the methods and compositions of this disclosure use one or more ATM kinase inhibitors to treat subjects having neurological disorder. In some embodiments, the ATM kinase inhibitor is any one of KU-55933, Dactolisib (BEZ235), KU-60019, JU-59403, AZ31, AZ32, AZD0156, AZD1390, VE-821, Wortmannin, Torin 2, CP-466722, Berzosertib (VE-822), or the like. In some embodiments, the one or more ATM kinase inhibitors include AZD1390. In other embodiments, the one or more ATM kinase inhibitors include KU60019.

In some embodiments, the neurological disorder comprises a neurodegenerative disease. In one embodiment, the neurodegenerative disease is any one of Alzheimer's disease, Mild Cognitive Impairment, Pick's disease, Parkinson's disease, Huntington's disease, and a prion-associated disease. In certain embodiments, the neurodegenerative disease comprises Parkinson's disease.

### Treatment

Also disclosed herein are methods of treating a neurological disorder by administering a subject in need a thereapeutically effective amount of an agent that inhibits the activity of ATM. In some embodiments, one or more of the agents described above is used in the provided methods.

In performance of these methods, the present disclosure further provides for pharmaceutical compositions comprising effective amounts of the foregoing agents/compounds (e.g., ATM kinase inhibitor(s)), separately or in combination with another therapeutic agent, in a suitable pharmaceutical carrier. The foregoing agents/compounds may be administered orally, intravenously, subcutaneously, intramuscularly, intranasally, intrathecally, or by other methods, several of which are known in the art, as would be appropriate for the chemical properties of the compound. It will be apparent to a person of ordinary skill in the art to determine the appropriate method of delivery of the foregoing agents/compounds.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

A pharmaceutical composition of this disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of this disclosure is contemplated. Supplementary active compounds can also be incorporated into the compositions.

The pharmaceutical compounds of this disclosure may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of this disclosure are dictated by and directly dependent on (a) the unique characteristics of the active compound and the particular therapeutic effect to be achieved, and (b) the limitations inherent in the art of compounding such an active compound for the treatment of sensitivity in individuals.

In some embodiments, the pharmaceutical composition may be administered with one or more additional therapeutic agents (e.g., an anti-Parkinson's disease such as carbidopa-levodopa, inhaled carbidopa-levodopa, carbidopa-levodopa infusion, catechol O-methyltransferase (COMT) inhibitors, anticholinergics, amantadine, and the like). In such embodiments, the one or more additional therapeutics may be administered prior to, concurrently with, or after the ATM kinase inhibitor.

As used throughout, “effective amount,” or “therapeutically effective amount,” refers to an amount sufficient to effect beneficial or desirable biological and/or clinical results. The effective amount of any of the therapeutic agents described herein can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Other factors that influence dosage can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

As used herein, administer or administration refers to the act of introducing, injecting or otherwise physically delivering a substance as it exists outside the body into a subject, such as by mucosal, intradermal, intravenous, intratumoral, intramuscular, intrathecal, intracranial, intrarectal, oral, subcutaneous delivery and/or any other method of physical delivery described herein or known in the art.

Any of the therapeutic agents described herein are administered in a number of ways depending on whether local or systemic treatment is desired, and on the area to be treated. The compositions are administered via any of several routes of administration, including orally, parenterally, intrathecally, intracranially, intramucosally, intravenously, intraperitoneally, intraventricularly, intramuscularly, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539. In some methods, the therapeutic agent can be delivered to the subject by way of an implantable device based on, e.g., diffusive, erodible, or convective systems, osmotic pumps, biodegradable implants, electrodiffusion systems, electroosmosis systems, vapor pressure pumps, electrolytic pumps, effervescent pumps, piezoelectric pumps, erosion-based systems, or electromechanical systems. Effective doses for any of the administration methods described herein can be extrapolated from dose-response curves derived from in vitro or animal model test systems.

Any of the therapeutic agents described herein can be formulated as a pharmaceutical composition. In some embodiments, the pharmaceutical composition can further comprise a carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Depending on the intended mode of administration, a pharmaceutical composition comprising a therapeutic agent described herein, can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein the terms “treatment”, “treat”, or “treating” refers to a method of reducing one or more of the effects of the disease or one or more symptoms of the disease, for example, AR, in the subject. Thus, in the disclosed methods, treatment can refer to a 10%, 20%, 30%, 40%, 50%, 60%, 70%, 80%, 90%, or 100% reduction in the severity of AR. In addition to alleviation or prevention of symptoms, treatment can also slow or stop the progression or worsening of a disease, disorder, or condition and/or the remission of the disease, disorder or condition. For example, a method for treating AR is considered to be a treatment if there is a 10% reduction in one or more symptoms of AR in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease or symptoms of the disease.

### Kits

Any of the methods provided herein can also be performed by use of kits that are described herein. Provided herein is a kit comprising agents for detection of the mutations in LRRK2 gene. Such agents may include primers for amplifying DNA, sequencing primers and enzymes that can be used in the PCR reactions or sequencing reactions.

In some embodiments, the kit further comprises at least one pharmaceutical composition comprising one or more ATM inhibitors as described above.

In some embodiments the kit further comprises components for detection of mitochondrial DNA damage including, for example, those disclosed in U.S. Pat. No. 11,001,890. In some embodiments, the agents include the primers and DNA polymerase for performing QPCR to detect the mtDNA.

In some embodiments, the kit further comprises instructions on how to use the kit to detect mutations in LRRK2 gene, mitochondria DNA damage, and/or administer the ATM inhibitors.

The kits can include components for isolating and/or detecting DNA in essentially any sample (e.g., urine, blood, etc.), and a wide variety of reagents and methods are, in view of this specification, known in the art. Hence, the kits can include vials, swabs, needles, syringes, labels, pens, pencils, or combinations thereof.

In some embodiments, commercially available components can also be included in the kits. For example, the kit can include components from QIAGEN, which manufactures a number of components for DNA isolation.

The kits can also include any of the following components: materials for obtaining a sample, enzymes, and buffers.

One of skill in the art would, in view of this specification, readily understand many combinations of components that a kit of the invention may comprise.

### Exemplary Embodiments

This disclosure provides the following nonlimiting exemplary embodiments.

Embodiment 1. A method of treating a neurological disorder comprising administering to the subject a therapeutically effective amount of an agent that inhibits the activity of ATM such that the neurological disorder is treated.

Embodiment 2. A method of rescuing LRRK2 genome instability in a subject suffering from a neurological disorder, the method comprising administering to the subject a therapeutically effective amount of an agent that inhibits the activity of ATM such that LRRK2 genome stability is rescued.

Embodiment 3. A method as in any of the preceding embodiments in which the neurological disorder comprises a neurodegenerative disease.

Embodiment 4. The method according to embodiment 3 in which the neurodegenerative disease is any one of Alzheimer's disease, Mild Cognitive Impairment, Pick's disease, Parkinson's disease, Huntington's disease, and a prion-associated disease.

Embodiment 5. The method according to embodiment 4 in which the neurodegenerative disease comprises Parkinson's disease.

Embodiment 6. The method as in any of the preceding embodiments in which the ATM kinase inhibitor is any one of KU-55933, Dactolisib (BEZ235), KU-60019, JU-59403, AZ31, AZ32, AZD0156, AZD1390, VE-821, Wortmannin, Torin 2, CP-466722, Berzosertib (VE-822), or the like.

Embodiment 7. The method according to embodiment 7 in which the ATM kinase inhibitor comprises AZD1390.

Embodiment 8. The method according to embodiment 8 in which the ATM kinase inhibitor comprises KU60019.

Embodiment 9. A kit comprising one or more ATM kinase inhibitors, and primers for detecting an LRRK2 gene encoding an LRRK2 variant, wherein the LRRK variant has increased kinase activity relative to the wild type LRRK2.

Embodiment 10. The kit of embodiment 9, wherein the LRRK2 variant has one or more mutations of G2019S, R1441C, R1441G, R1441H, Y1699C, or I2020T.

Embodiment 11. A method of treating a neurological disorder comprising administering to the subject a therapeutically effective amount of an ATM inhibitor.

Embodiment 12. The method of embodiment 11, wherein the subject expresses a LRRK2 variant protein, wherein the LRRK2 variant protein has increased kinase activity as compared to a wild type LRRK2 protein.

Embodiment 13. The method of embodiment 12, wherein the LRRK2 variant protein comprises one or more amino acid substitutions relative to SEQ ID NO: 1.

Embodiment 14. The method of embodiment 12, wherein the one or more amino acid substitutions comprise one or more of G2019S, R1441C, R1441G, R1441H, Y1699C, or I2020T.

Embodiment 15. The method of embodiment 12, wherein the LRRK2 variant protein has an amino acid sequence that is at least 90% identical to SEQ ID NO: 2 or SEQ ID NO: 3 and comprises one or more of G2019S, R1441C, R1441G, R1441H, Y1699C, or I2020T.

Embodiment 16. The method as in any of the preceding embodiments, wherein brain cells of the subject have a higher level of mitochondria DNA damage as compared to brain cells of a control subject.

Embodiment 17. The method of embodiment 16, wherein the mitochondria DNA damage is oxidized mtDNA lesions or presence of abasic sites in the mitochondria DNA.

Embodiment 18. The method of embodiment 16, wherein the brain cells are dopaminergic neurons of the subject.

Embodiment 19. The method of embodiment 16, wherein the brain cells are located in the ventral midbrain region.

Embodiment 20. The method of any one of the preceding embodiments, wherein the neurological disorder comprises a neurodegenerative disease.

Embodiment 21. The method according to embodiment 20, wherein the neurodegenerative disease is any one of Alzheimer's disease, Mild Cognitive Impairment, Pick's disease, Parkinson's disease, Huntington's disease, or a prion-associated disease.

Embodiment 22. The method according to embodiment 21 in which the neurodegenerative disease comprises Parkinson's disease.

Embodiment 23. The method of any one of embodiments 11-22 in which the ATM kinase inhibitor is any one of KU-55933, Dactolisib (BEZ235), KU-60019, JU-59403, AZ31, AZ32, AZD0156, AZD1390, VE-821, Wortmannin, Torin 2, CP-466722, Berzosertib (VE-822), and the like.

Embodiment 24. The method according to embodiment 23 in which the ATM kinase inhibitor comprises AZD1390.

Embodiment 25. The method according to embodiment 24 in which the ATM kinase inhibitor comprises KU60019.

Embodiment 26. A method of rescuing LRRK2 genome instability in a subject suffering from a neurological disorder, the method comprising administering to the subject a therapeutically effective amount of an agent that inhibits the activity of ATM such that LRRK2 genome stability is rescued. The LRRK2 genome instability is due to the presence of one or more mutations in the LRRK2 gene that result in expression of a LRRK2 variant protein as described above.

Embodiment 27. The method of embodiment 26, wherein rescue of LRRK2 genome stability is indicated by a DNA lesion frequency in the subject that is substantially identical to the DNA lesion frequency in a control subject that expresses a wild type LRRK2 protein.

Embodiment 28. The method of any one of embodiment 11-27, wherein the method further comprises administering one or more additional therapeutic agents.

Embodiment 29. A kit comprising: (a) primers for detecting a genomic sequence encoding a LRRK2 variant protein in a biological sample, wherein the LRRK2 variant protein has increased kinase activity relative to wild type LRRK2 and instructions for identifying the genomic sequence as encoding the LRRK2 variant protein.

Embodiment 30. The kit of embodiment 29, wherein the LRRK2 variant protein has one or both of a G2019S mutation or a R1441C mutation.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The following Examples are provided by way of illustration and not by way of limitation.

### EXAMPLES

### Example 1. Mitochondrial DNA Damage Detection Assay

This example describes how mitochondrial DNA damage was measured using a quantitative PCR-based assay as described above. The following reagents and protocol steps as disclosed in Sanders et al., Curr Protoc Toxicol. 2018 May; 76(1): e50, doi: 10.1002/cptx.50 and also in U.S. Pat. No. 11,001,890. The entire contents of said publication and patent are herein incorporated by reference.

Reagents

Primers were purchased from Integrated DNA Technologies; primer sequences and annealing temperatures are available in (C. P. Gonzalez-Hunt et al., 2016). Other reagents include Bovine Serum Albumin (BSA; Gemini Bio Products); Nuclease Free Water (e.g., Sigma-Aldrich W4502-1L); High-quality thermal cycler (we use the Biometra T1); 0.2 mL PCR tubes (individual tubes, strips, or sealed PCR plates are acceptable). using the KAPA LongRange Hot Start DNA polymerase

Prepare Master Mix:

Make a master mix if several samples are being run simultaneously. Use the KAPA LongRange Hot Start kit (Millipore Sigma, Darmstadt, Germany) and add the following components in this order: Nuclease-free H2O (for a final volume of 50 μL); 15 ng sample DNA with final target template DNA concentration of 3 ng/μL (typically, it is possible to reduce this to as low as 5 ng input (concentration of 1 ng/μL), especially if you have a PCR-based absolute measure of mitochondrial and nuclear DNA copy number for normalization purposes); 10 μL of 5×buffer solution; 1 μL of BSA in nuclease-free H₂O (1.0 mg/mL stock); 1 μL of dNTPs (10 mM stock); 2.5 μL of each primer working solution (10 μM stock); 3.5 μL of MgCl2 (25 mM stock supplied in kit).

Briefly (1-2 seconds) vortex and spin (˜5 seconds, using a mini-centrifuge) the master mix, and aliquot 25 μL into the appropriate number of PCR reaction tubes (or wells of a PCR plate).

Perform PCR

Perform the PCR reaction using the PCR amplification profile is as follows for the large mitochondrial PCR product using the KAPA LongRange Hot Start kit:
Melting: 94° C. for 3 min
Amplification: the optimized number of cycles (26-28) of melting (94° C. for 15 sec) and annealing (66° C. for 12 min).
Final extension: To complete the profile and finalize amplification of products, perform a final extension for 10 min at 72° C.
Holding: Hold at 4° C. until products are removed for quantification. If the post-amplification holding step will be performed overnight, or for other reasons the samples are likely to sit in the thermocycler for an extended period of time, it is preferable to hold at 8° C., as this temperature puts less strain on the thermocycler.

Quantify the Resulting PCR Products

(1) Add 10 μL of each PCR product and 90 μL of TE buffer to each of triplicate wells. This amount may be adjusted to ensure that readings are well within the standard curve (we often use 5 μL), but the amount pipetted should not be so low as to significantly increase pipetting error (e.g., 1 μL); (2) Add 100 μL PicoGreen working solution to each well and incubate at room temperature in the dark for 10 min. Follow steps 6, 7 and 8 in Support Protocol 2 for fluorescent quantification of DNA.

Perform Data Analysis:

After obtaining the fluorescence values from the plate reader software, subtract the no template control values and/or background fluorescence from PCR product values.

Average all sample values of the triplicate wells. If the “50% control” does not fall within 40%-60% of the untreated controls, this data set is invalid and cycle number should be adjusted.

Normalize large mitochondrial PCR product fluorescence values for copy number differences using the small mitochondrial PCR product. To normalize, divide each sample's small mitochondrial product value by the average of all small mitochondrial products to get a correction factor for each sample. Then, divide each sample's large mitochondrial value by its correction factor. This is the normalized large mitochondrial fluorescence value (A. M. Furda, Bess, Meyer, & Van Houten, 2012a).

Alternatively, real-time PCR-derived mtDNA and nDNA copy number values can be used for normalization; see reference (Rooney et al., Methods Mol Biol. 2015; 1241:23-38. doi: 10.1007/978-1-4939-1875-1_3) for real-time PCR protocol.

Divide the normalized fluorescence values of each sample by the average normalized fluorescence value to get a ratio.

Perform the negative natural log (−ln) of the ratio to obtain the lesion frequency per fragment. This value is normalized to the number of lesions/10 kb. Examples of this calculation can be found in (C. P. Gonzalez-Hunt et al., 2016).

At least three biological samples should be analyzed per condition, with at least three separate QPCR runs to calculate the average lesion frequency.

### Example 2. mtDNA Damage is Increased with LRRK2 Mutations and Idiopathic PD

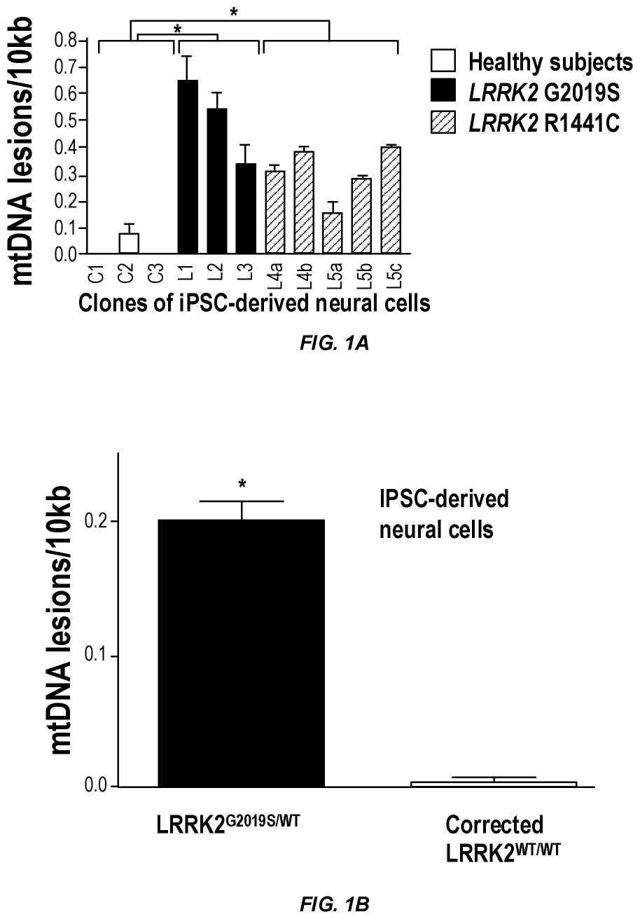

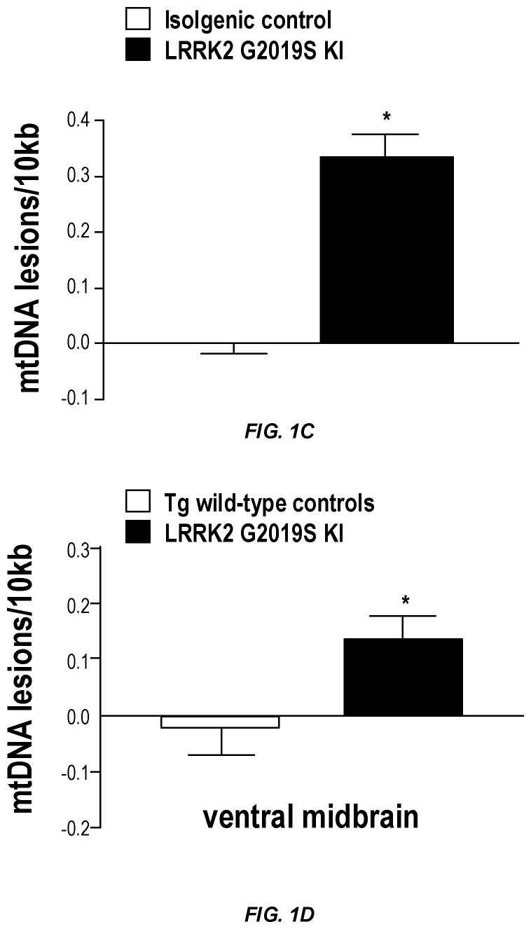

Employing the quantitative PCR based mtDNA damage assay described in Example 1, we recently discovered that human induced pluripotent stem cell (iPSC)-derived dopaminergic (DA) neurons from individuals carrying LRRK2 mutations demonstrate significant levels of mtDNA damage (FIG. 1A).

When the G2019S mutation was ‘corrected’ with gene editing, mtDNA damage was no longer detectable (FIG. 1B). We further substantiated that LRRK2 G2019S causes mtDNA damage, using LRRK2^{G2019S/G2019S} knock-in (KI) human embryonic kidney 293 (HEK293) cells obtained by CRISPR/Cas9 gene editing, in which we found significantly increased mtDNA damage (FIG. 1C). LRRK2 G2019S knock-in (GKI) mice have also been generated and demonstrate dopaminergic deficits and mitochondrial alterations. This is a valuable model to understand early pathophysiological effects of mutant LRRK2. We verified that mtDNA damage is increased specifically in the ventral midbrain in LRRK2 GKI mice compared to wild-type littermates (FIG. 1D). Thus, the mtDNA damage phenotype can be unambiguously attributed to the LRRK2 G2019S mutation.

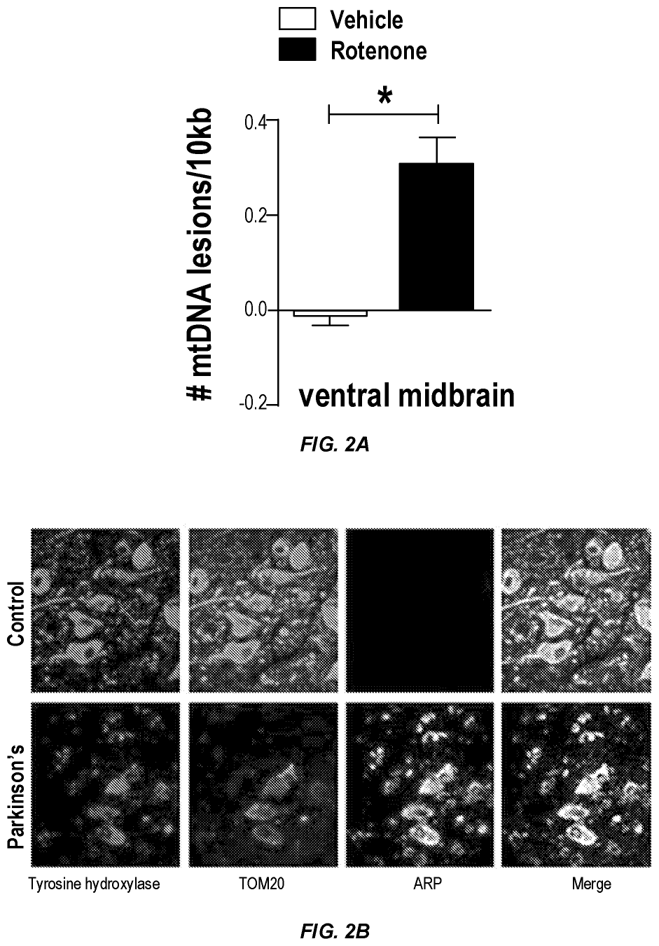

The etiology of PD is attributed to both genetic and environmental factors. To explore the effect of environmental factors, we chose the systemic rotenone model, as it mimics many of the clinical and pathological features of idiopathic PD in an animal model. To study pathological events prior to neurodegeneration, we used a preclinical rotenone paradigm. We found that rats exposed to rotenone increased mtDNA damage in the ventral midbrain (FIG. 2A).

Because there were no tools with which to examine specific kinds of mtDNA damage in intact tissue and in a cell-specific manner, we developed a novel histochemical technique to detect a specific form of mtDNA damage—abasic sites. We focused on abasic sites because the inability to effectively repair abasic sites can compromise mitochondrial function and contribute to neurodegeneration. Applying this assay, we were the first to identify the molecular identity of mtDNA damage to be abasic sites in substantia nigra dopamine neurons from postmortem PD brains (FIG. 2B); similar results were found in rotenone treated rats. The persistence of abasic sites suggests an ineffective base excision repair (BER) response in PD. These results indicate that mtDNA damage is detectable prior to any signs of degeneration, suggesting mtDNA damage occurs early in PD pathogenesis—and persists in midbrain neurons under conditions of mitochondrial impairment and in human PD. Taken together, our data from LRRK2, rotenone and human PD postmortem brains illustrate that mtDNA damage is a common feature of both genetic (LRRK2) and idiopathic PD and may play a causal role in neurodegeneration.

### Example 3. mtDNA Damage is Reversed by LRRK2 Kinase Inhibition

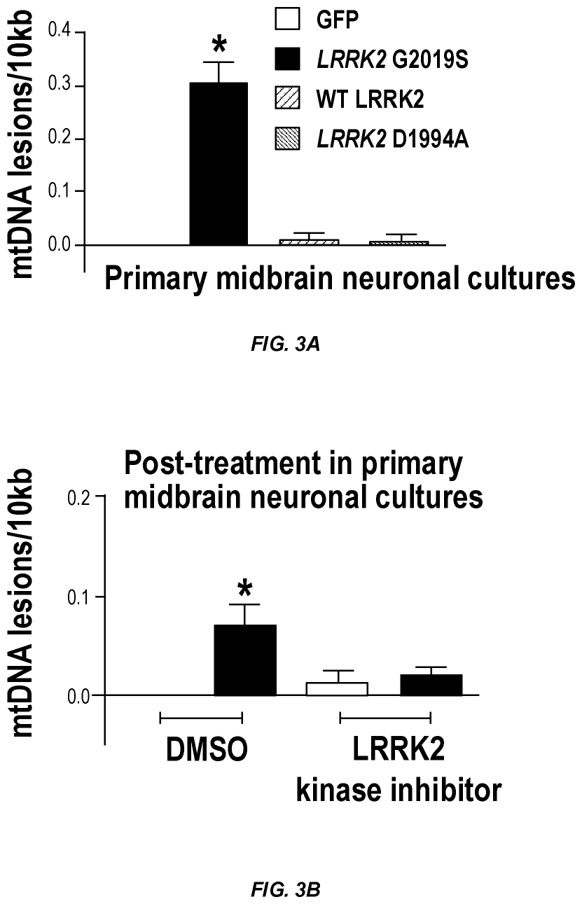

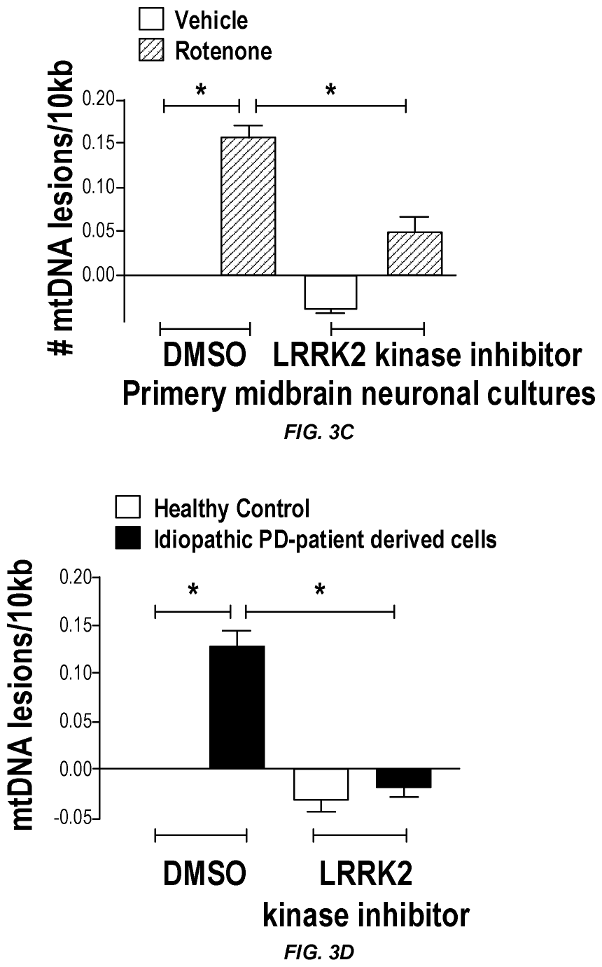

As pathogenic LRRK2 mutations are associated with a kinase gain-of-function, great efforts have focused on the development of potent and selective inhibitors of LRRK2 kinase activity. While treatment with LRRK2 kinase inhibitors both in vitro and in vivo have demonstrated an ability to mitigate toxicity and neurodegeneration, the mechanism(s) of action by which LRRK2 inhibitors provide therapeutic benefit are unknown. Thus, first we tested whether mutant LRRK2 mediated mtDNA damage is kinase dependent. We found that mtDNA damage was significantly increased in primary midbrain neurons expressing LRRK2 G2019S (increased kinase activity mutant) relative to GFP, LRRK2 wild-type, or LRRK2 D1994A (kinase dead) mutant expressing cultures (FIG. 3A).

Treating with a LRRK2 kinase inhibitor either prior to, concurrently, or following the presence of mtDNA damage was able to restore mtDNA damage to control levels (FIG. 3B). Similar results were found using PD patient-derived cells to evaluate the applicability of these findings in the setting of endogenous LRRK2 levels. Exposure of LRRK2 G2019S patient-derived cells to LRRK2 kinase inhibition also reversed increased mtDNA damage to healthy control levels within 24 h. Secondly, we tested whether increased mtDNA damage in a rotenone model and idiopathic PD is kinase dependent. LRRK2 kinase inhibition prior to rotenone exposure prevented mtDNA damage in primary midbrain neurons (FIG. 3C). Exposure of idiopathic PD patient-derived cells to LRRK2 kinase inhibition reversed mtDNA damage to healthy control levels (FIG. 3D). Overall, LRRK2 kinase activity is playing a critical role in mtDNA damage in both LRRK2 and idiopathic PD.

### Example 4. mtDNA Base Excision Repair (BER) Activity is Decreased in LRRK2 PD

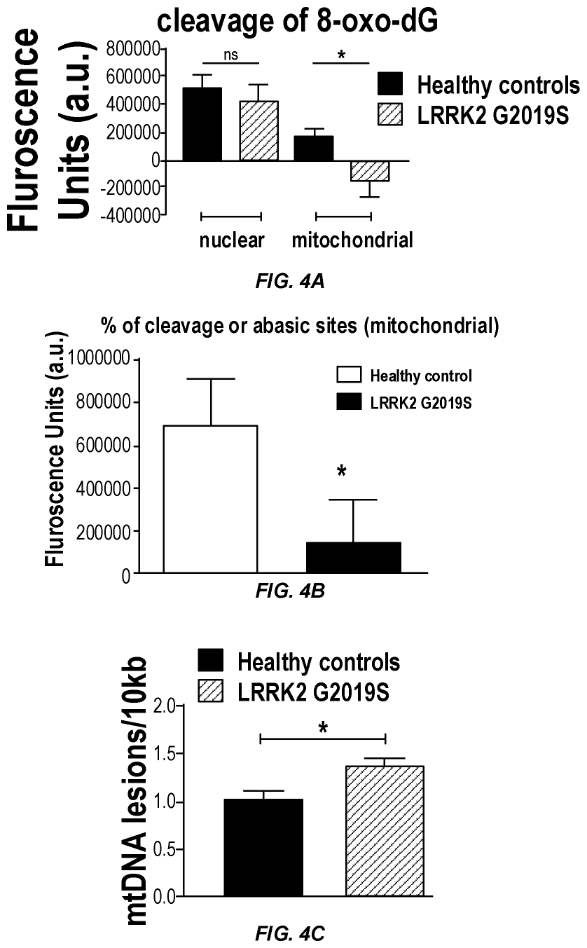

The persistence of abasic sites suggests an ineffective base excision repair (BER) response in PD. Historically mitochondrial DNA repair mechanisms have been considered to be low efficiency or non-existent relative to nuclear DNA repair—but current data disputes this notion and instead acknowledges robust oxidative mtDNA repair. BER is the chief DNA repair pathway to repair oxidative DNA damage and new mechanisms and players are still being discovered. Given our observations of increased mitochondrial abasic sites in models and human PD, taken together with our evidence that BER variants and mitochondrial dysfunction- or oxidative stress-related pesticide exposure increase human PD risk, we determined mitochondrial BER activity in LRRK2 patient-derived cells. The Glyco-SPOT DNA Repair Assay is a multiplexed fluorescent Oligonucleotide Cleavage Assay used to simultaneously evaluate the cleavage efficiency of glycosylases and AP endonucleases that participate in BER (LXRepair). The ExSy-SPOT is similar but quantifies excision/synthesis of BER lesions in a single biochip. Mitochondrial extracts were prepared in a buffer and protocol already optimized in partnership with LXRepair (FIGS. 4A, 4B). Mitochondrial extracts from healthy controls and LRRK2 G2019S patient-derived lymphoblastoid cells under basal conditions were evaluated on the Glyco-SPOT and ExSy-SPOT DNA Repair Assay. The mitochondrial (but not nuclear) excision activity of 8-oxo-dG and abasic sites were found to be decreased in LRRK2 G2019S patient-derived cells (FIGS. 4A, 4B).

The detection of mtDNA damage is currently hindered by the lack of appropriate or sensitive tools. The Sanders Laboratory has made considerable advances in successfully developing a PCR-based method to measure mtDNA damage, as disclosed in U.S. Pat. No. 11,001,890. This method is based on the principle that cleaving DNA with an enzyme (e.g., FPG, which selectively releases damaged bases such as 8-oxoG and Fapy-G from DNA) will render the DNA resistant to PCR amplification. 8-oxoguanine is one of the most common DNA lesions resulting from reactive oxygen species modifyin guanine. FapyG stands for 2,6-diamino-4-hydroxy-5-formamidopyrimidine, a ring-opened lesion that forms when hydroxyl radicals attack guanine, followed by one-electron reduction of the hydroxyl adduct radicals. Upon treatment with the glycosylase FPG, for example, oxidative base lesions caused by mutant LRRK2 will be processed and cleaved by the glycosylase, resulting in a 1 base gap. This will make the DNA less amenable to PCR amplification, resulting in a shift in the amplification curve towards the right (increase cycle threshold). PCR amplification can then be compared in treated versus untreated DNA. We have now further improved our assay and can detect specific kinds of oxidative mtDNA damage (FIG. 4C). Using this innovative approach, we found higher levels of oxidized mtDNA damage (types recognized by BER) in LRRK2 G2019S patient-derived cells compared to controls (FIG. 4C). Importantly, these findings are consistent with the types of mtDNA repair defects described in FIGS. 4a and b, indicating that specific oxidative mtDNA lesions repaired by BER accumulate with mutant LRRK2 G2019S.

### Example 5. Activation of the ATM Mediated DNA Damage Response Pathway

We found that basal levels of ATM pSer1981 were increased in both an in vitro and in vivo LRRK2 G2019S model: LRRK2^{G2019S/G2019S} KI HEK293 cells and ventral midbrain derived from LRRK2 GKI mice (FIGS. 5A-5D). Rotenone exposure also increased levels of ATM pSer1981 (FIGS. 5E, 5F). Consistent with our data, a quantitative phosphoproteomics screen identified ATM pSer1981 in LRRK2 G2019S cells and activated ATM is detected in other PD model systems.

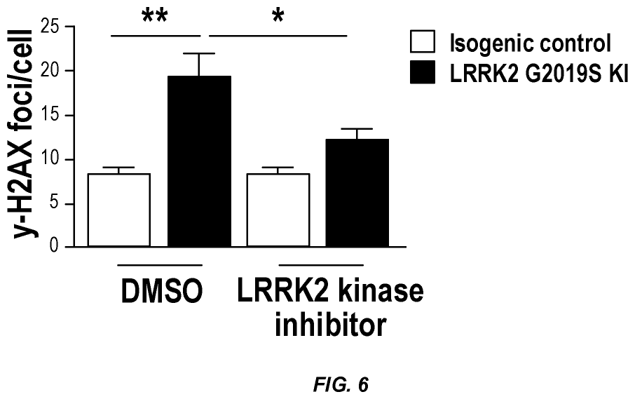

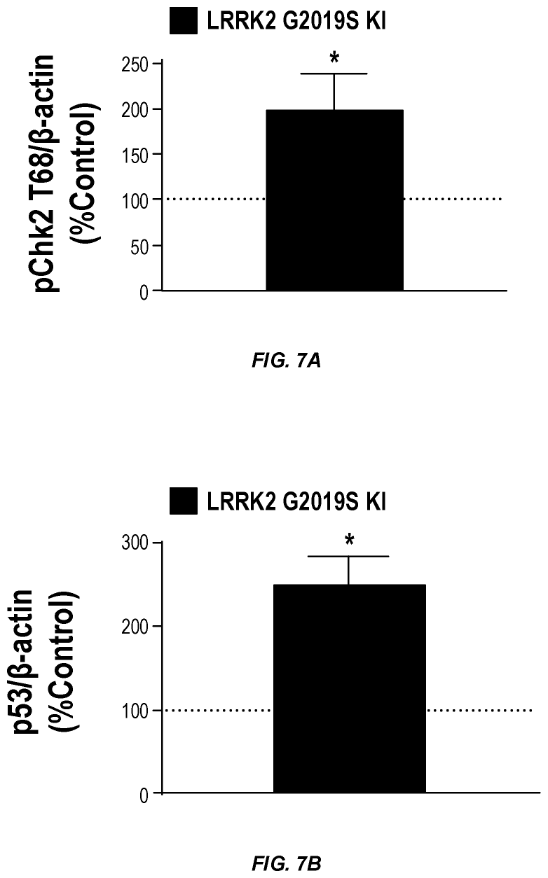

Whether LRRK2 kinase activity has broad impact on nuclear genome integrity is unknown. DNA double-strand breaks (DSBs) in the nucleus trigger the phosphorylation of 5139 in the C-terminal of histone variant H2AX (typically named γ-H2AX). ATM primarily mediates γ-H2AX phosphorylation. Consistent with ATM activation, we observed significantly increased γ-H2AX foci using LRRK2^{G2019S/G2019S} KI cells compared to isogenic wild-type control cells, highlighting that an ATM substrate is also increased (FIG. 6). Strikingly, acute LRRK2 kinase inhibition reversed γ-H2AX foci (FIG. 6) which implicates ATM downstream of mutant LRRK2. As used herein, the term “acute,” when referring to a treatment or procedure, means said treatment or procedure is completed in a time frame of 2 hours or less. ATM-mediated activation of the DNA damage response pathway includes phosphorylation of downstream effectors such as pChk2 and p53. Both pChk2 and p53 are increased in LRRK2^{G2019S/G2019S} KI cells compared to isogenic wild-type control cells (FIGS. 7A, 7B). Collectively these data substantiate that the ATM-mediated DNA damage response pathway has been up-regulated with the LRRK2 G2019S mutation.

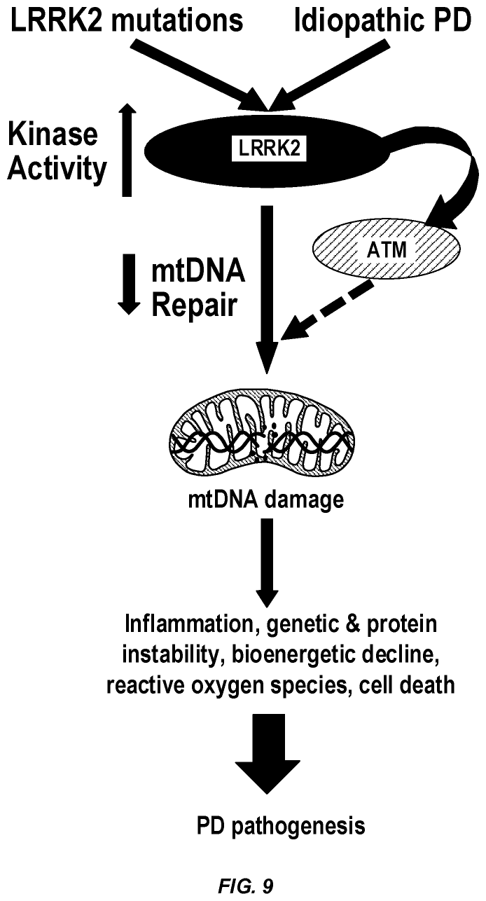

In vitro experiments have shown that LRRK2 G2019S was able to phosphorylate peptides derived from ATM. To further determine whether ATM is a putative LRRK2 substrate in cells, we generated an antibody to LRRK2's consensus phosphorylation predicted site on ATM. ATM pSer1769 appeared increased in LRRK2^{G2019S/G2019S} KI HEK293 cells. ATM is the kinase that mediates γ-H2AX phosphorylation in mutant LRRK2 cells (FIGS. 9A, 9B) and pharmacological inhibition of ATM kinase activity reversed mtDNA damage in LRRK2^{G2019S/G2019S} KI cells (FIGS. 9A, 9B). These exciting results suggest that ATM is downstream of mutant LRRK2, pathogenic LRRK2 mutations activate ATM and increase a novel phosphorylated form of ATM (possibly directly), potentially regulating the DNA damage response pathway in a PD context. Overall, these results provide the foundation for a functional relationship between LRRK2 and ATM.

### Example 6. Proposed Experiments

These findings above are the basis for our working model for the control of genome integrity by LRRK2 (FIG. 9): dysfunctional LRRK2 triggers the ATM-mediated DNA damage response pathway, which impairs mtDNA repair capacity, leading to an increase in mtDNA damage.

Towards the verification of this working model, we will test the following hypotheses: a) Increased oxidized mtDNA lesions are dependent on increased LRRK2 kinase activity in both genetic and idiopathic PD (Aim 1). b) Mitochondrial DNA repair capacity, in particular the early steps of the mtDNA BER repair pathway, are impaired in PD (Aim 2). c) Targeting activation of the ATM mediated DNA damage response pathway will attenuate PD-associated pathology (Aim 3).

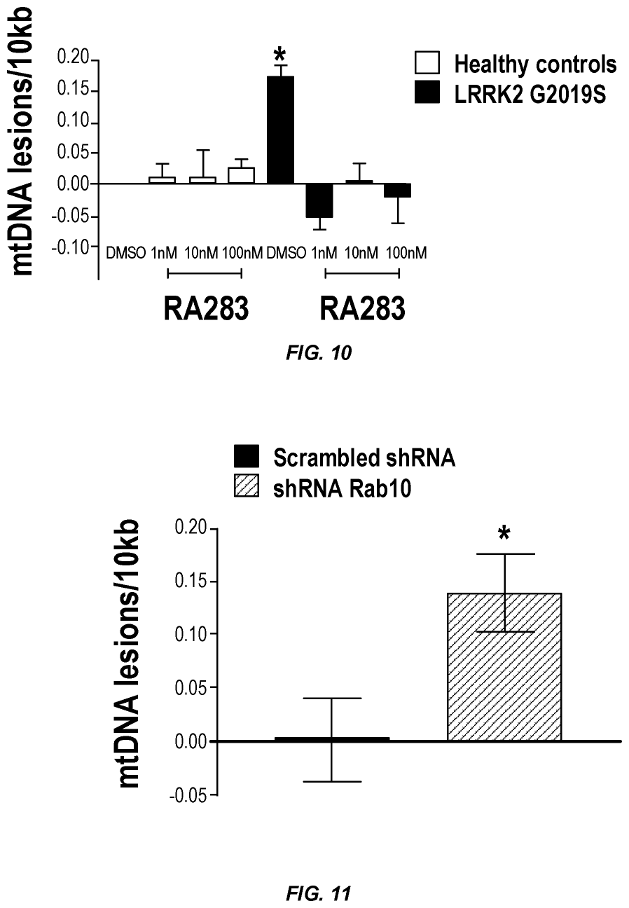

For Aims 1 & 2 of this proposal, we will employ LRRK2 and idiopathic PD patient-derived iPSC-derived dopaminergic (DA) neurons, with isogenic and age-matched controls to better understand neuronal cell-type specific roles of LRRK2. These mature DA neurons can be characterized by expression of a mature neuronal marker TUBB3 and dopaminergic (DA)-specific markers. The DA-specific markers include TH and GFAP. In one exemplary experiment, 46.9% cells in the mature DA neuron population (derived from iPSC) were posive for either GFAP or TH, and 28.3% of cells were positive for both TUBB3 and TH. See U.S. Provisional Application No. 63/076,505, and FIG. 10.

To ensure a high degree of rigor and data reproducibility, all conditions involving iPSC-derived neurons will be conducted with a minimum of three experimental replicates with each biological iPSC line, three of which will be obtained from each donor; see Table 1 below.

TABLE 1
iPSC cell lines obtained from donors

| Subject ID | Mutation | Source | Sex | Phenotype |
|---|---|---|---|---|
| GM23338 | n/a | Coriell | M | HC |
| WTSIi143-A | n/a | EBiSC | M | HC |
| BIONi037-A | n/a | EBiSC | F | HC |
| NDS00010 | G2019S | NINDS | M | PD |
| NDS00010-C | Isogenic | NINDS | M | HC |
| | control | | | |
| NDS00210 | G2019S | NINDS | M | PD |
| NDS00210-C | Isogenic | Sanders Lab | M | HC |
| | control | | | |
| UOXFi007-A | G2019S | EBiSC | F | PD |
| UOXFi007-A-C | Isogenic | Sanders Lab | F | HC |
| | control | | | |
| NDS00137 | G2019S | NINDS | M | At-risk |
| NDS00138 | G2019S | NINDS | M | At-risk |
| NDS00104 | R1441G | NINDS | F | PD |
| NDS00104-C | Isogenic | Sanders Lab | F | HC |
| | control | | | |
| NDS00205 | R1441C | NINDS | M | At-risk |
| NDS00204 | R1441C | NINDS | F | At-risk |
| NDS00083 | n/a | NINDS | M | PD |
| | (Idiopathic) | | | |
| ESi002-A | n/a | EBiSC | F | PD |
| | (Idiopathic) | | | |
| ESi001-A | n/a | EBiSC | M | PD |
| | (Idiopathic) | | | |

Table 1 shows iPSC cell lines to be used in these experiments. The lines are age-matched. We will also take advantage of new lines as they become available through PPMI at MIFF and other sources. Since no isogenic R1441G lines exist, this will be the first isogenic control regenerated and will be made freely available to the scientific community.

A minimum of three independent rounds of differentiation per iPSC line, with all lines simultaneously induced to neural progenitor cells will allow for direct comparisons and reduced variability. Cell lines were chosen to include males and females in each group, but sex will not be evaluated as an experimental variable. Raw, non-normalized data will be presented in publications whenever possible. For statistical analyses we will use the Student's t-test or ANOVA with Tukey's post hoc analysis as appropriate.

Aim 1. To determine the Role of Dysfunctional LRRK2 Activity in Mitochondrial Genome Integrity

Rationale: Since the discovery of LRRK2, considerable efforts have gone into uncovering its fundamental cellular function. Although LRRK2 has been implicated in a variety of biological processes, the basic function(s) of this protein and the effects of disease-causing mutations have remained poorly understood. The majority of the validated mutations in LRRK2 known to cause pathogenicity are found in the kinase domain (G2019S, I2020T) or GTPase domain (R1441C/G/H). Despite the shared PD clinical manifestations of mutations found in either the kinase or GTPase domain, biochemical differences of the various LRRK2 mutations (e.g., between G2019S and R1441 sites) are now better appreciated. A comparison of the pathogenic mechanisms between LRRK2 G2019S vs the R1441 site may be informative, as mutations in the GTPase domain show greater penetrance and may, therefore, exhibit enhanced pathology. While our published and preliminary data have primarily focused on the LRRK2 G2019S mutation, we have found that mtDNA damage is also increased in iPSC-derived DA neurons from LRRK2 R1441C carriers (FIGS. 3A-3D). Interestingly in addition to LRRK2 mutations, we found increased mtDNA damage in environmental toxicant-induced PD models and in idiopathic PD. Understanding the role that mtDNA damage plays in the pathogenesis of PD necessitates specifying the molecular identity of the particular type(s) of mtDNA damage that has a propensity to accumulate in diseased neurons.

Though LRRK2 mutations and idiopathic PD both demonstrate an increase in mtDNA damage, whether there is a shared underlying mechanism(s) that includes LRRK2 kinase activity has not been explored. Unexpectedly, we observed mtDNA damage in peripheral cells derived from idiopathic PD subjects was also reversed by LRRK2 kinase inhibition (FIG. 3), but is not clear whether these findings would apply to neurons. Furthermore, it is unknown whether our preliminary findings showing the increase in mtDNA damage or the dependency on LRRK2 kinase activity extends to at-risk LRRK2 carriers—crucial to identifying potential early pathogenic events for intervention. Therefore, the overall goal of Aim 1 is to mechanistically define the role of LRRK2 kinase activity in the formation of specific mtDNA lesions. To do this, we will utilize multiple well-established in vitro cellular PD models that include (1) an endogenous model—LRRK2^{G2019S/G2019S} or LRRK2^{R1441C/R1441C} knock-in HEK293 cells generated by CRISPR/Cas9 gene editing and isogenic controls, and (2) LRRK2 mutant patient-derived and at-risk individuals carrying LRRK2 mutations or idiopathic PD iPSC-derived DA neurons (see Table 1 for full list of iPS-cell lines).

Experimental Design Aim 1A: Our hypothesis is that specific oxidative mtDNA lesions accumulate in PD diseased neurons. The goal of Aim 1A is to determine the abundance and composition of mtDNA lesions in LRRK2 mutation-associated PD and in idiopathic PD. To do this, using PD patient-derived DA neurons and LRRK2 KI cells, we will utilize the PCR-assay as disclosed in U.S. Pat. No. 11,001,890 to detect specific kinds of mtDNA damage as described in the preliminary data section (FIGS. 4A-4C). Of note, standard assays used to identify nuclear DNA damage cannot be applied to mitochondrial DNA. Isolated DNA samples will be treated with BER enzymes, such as DNA with glycosylases (e.g. UDG or Fpg) and other enzymes such as APE1, FEN1, and T4 DNA ligase (all available from New England Biolabs). Cell lines that have been treated with different oxidative stressors (hydrogen peroxide etc.) and oligonucleotides with known DNA damage will serve as positive controls. To detect DNA single- and double-strand breaks, mitochondrial DNA will be isolated and subjected to alkaline agarose gels. Since increased mtDNA mutation burden has been reported in PD, these experiments will be performed in parallel with Duplex Sequencing (DS), currently the highest accuracy sequencing method, in order to determine whether LRRK2 or idiopathic induced mtDNA damage converts to an increase in mutagenesis. DS extends the idea of molecular barcoding by using double-stranded molecular barcodes to take advantage of the fact that the two strands of DNA contain complementary information. For duplex sequencing, sheared duplex DNA is ligated with a random, yet complementary, double-stranded nucleotide sequence and PCR amplified, which results in many copies derived from a single parental strand of DNA that share a common tag sequence. After sequencing, reads sharing the same tag are grouped and a consensus sequence is calculated for each family to create a single strand consensus sequence, which corresponds to an individual strand of DNA and filters out sequencing errors. Then, the complementary tags derived from the same original molecule are compared to each other to create a highly-accurate duplex consensus sequence read that filters out mutations present in one strand of DNA but not in the other. the error rate of DS is typically about 10⁻⁷, two orders of magnitude less than traditional sequencing technology based on single-stranded tagging, The objective of Aim 1B is to determine the dose-dependency the detection of a mutation in ≤10 sequenced bases.

Experimental Design 1B: Our preliminary data demonstrates that both a 24 hour (FIG. 3A-3D) and a 1.5 hour (FIG. 13) exposure reversed LRRK2 G2019S-induced mtDNA of mtDNA damage reversal and dynamics.

We will determine the time course and concentration-dependence of LRRK2 kinase inhibitors (such as MLi-2) that reverse the mtDNA phenotype. We will culture PD patient-derived DA neurons or LRRK2 mutant KI cells with a dose response curve with two distinct LRRK2 kinase inhibitors at various times (i.e. 1.5 hr, 6 hr, up to 24 hours). Target engagement will be verified by measuring LRRK2 pSer935. We will further test the rapid and dynamic nature of the mtDNA damage phenotype. Cells will be treated acutely with a LRRK2 kinase inhibitor, followed by a washout into media without inhibitor, to determine if the mtDNA damage phenotype is fully reestablished.

By the end of these studies we will distinguish the type of mtDNA damage that preferentially accumulates in PD neurons, in order to identify which specific mtDNA lesions may be central to the pathogenesis of the disease. Understanding the type(s) of mtDNA damage and mutational signatures will give us insight into the potential pathways that are defective, thereby beginning to elucidate the underlying causal mechanism of mtDNA damage. In particular, the mutation signatures will reveal a molecular “footprint” of the types of mtDNA damage that were repaired inefficiently or if they are replication mediated, delineating between dysregulated DNA repair or DNA replication. We expect to find increased oxidative mtDNA lesions known to be repaired by BER, since the brain is particularly susceptible to oxidative damage due to its high levels of polyunsaturated fatty acids and relatively low antioxidant activity.

However, it is possible the nature of accumulating mtDNA lesions could be due to sources other than oxidative damage, therefore alkylating lesions or bulky adducts will also be measured. While an increase in mtDNA damage is shared by genetic and idiopathic PD, the specific kinds of lesions may not be identical, which will yield critical information for the mechanistic basis of this damage, highlighting the possibility that different types of mtDNA damage are reflective of diverse disease pathologies which may need different therapeutic approaches.

Aim 2: To Define the Cellular Mechanism(s) of Regulating the Accumulation of Mitochondrial DNA Damage in LRRK2 and Idiopathic PD

Rationale: Multiple cellular pathways are important in maintaining mitochondrial homeostasis—including (but not limited to)—vesicular trafficking, mitophagy (the selective degradation of mitochondria by autophagy) and mtDNA repair. Rab GTPases are key regulators of vesicle-mediated transport and have been proposed to be a relevant pathway altered in PD. Pathological LRRK2 mutations lead to enhanced Rab phosphorylation and are reversed by LRRK2 kinase inhibition. LRRK2 is proposed to phosphorylate fourteen different Rab GTPases, including Rab10. It is currently unclear how phosphorylation of LRRK2 Rab substrates result in molecular events that lead to neurodegeneration. Interestingly, two recent studies link LRRK2 mutations, Rabs, mitochondria and mitophagy. One group found that Rab10 accumulates on depolarized mitochondria targeted for mitophagy and that LRRK2 G2019S-mediated phosphorylation impairs Rab10 mitochondrial recruitment and mitophagy. Our preliminary data suggest that reduced levels of Rab10 increased mtDNA damage, consistent with the previous report that Rab10 facilitates mitophagy in wild-type cells (FIG. 13).

It has also been reported that mitochondrially anchored Rab29 (a LRRK2 substrate and activator) instigates active LRRK2 accumulation and pRab 10 on mitochondria. Taken together with our preliminary evidence for a decrease in mtDNA repair capacity (FIG. 4), these results suggest connections between mtDNA repair, vesicular trafficking and mitophagy that promote dysfunctional mitochondria in PD. Importantly, our studies do not conflict with proposed cytoplasmic roles for LRRK2 in membrane/vesicular trafficking, but instead, intimate that these pathways are related. The relative contribution of each of these pathways to mtDNA damage associated with LRRK2 G2019S, R1441G/C mutations or in idiopathic PD is unknown—or whether defects in vesicular trafficking, mitophagy or mtDNA repair are detectable prior to the development of PD (i.e. at-risk subjects). In Aim 2, we will determine the role of multiple mitochondrial related pathways to the underlying mechanisms of mtDNA damage in human idiopathic and LRRK2 mutant patient-derived and at-risk individuals carrying LRRK2 mutations in iPSC-derived DA neurons (Table 1) and LRRK2^{G2019S/G2019S} or LRRK2^{R1441C/R1441C} knock-in HEK293 cells, to better understand the underlying mechanism(s) by which mitochondrial dysfunction occurs in PD.

Experimental Design 2A: We hypothesize that overexpression of the LRRK2 substrate Rab10 will rescue mtDNA damage levels in PD. The goal of Aim2A is to test whether mtDNA damage accumulation is mediated by Rab10 or involves other LRRK2 Rab substrates. LRRK2 Rab substrates (including but not limited to: Rab8a/b, Rab10, Rab12, Rab29, Rab35) will be modulated by viral mediated knockdown and overexpression of wild-type and phosphomimetic/phosphoresistant mutants in cultured PD patient-derived DA neurons and LRRK2 mutant KI lines (i.e. Rab10 T73E and T73A, respectively) and mtDNA damage assessed. Viral platforms will be generated. While Western blots will confirm silencing or overexpression of Rabs, immunocytochemistry will be performed in parallel to determine localization of Rab proteins. It will be further determined whether Rab10 and other identified Rabs mitigate mtDNA damage by effects on mitophagy as described below.

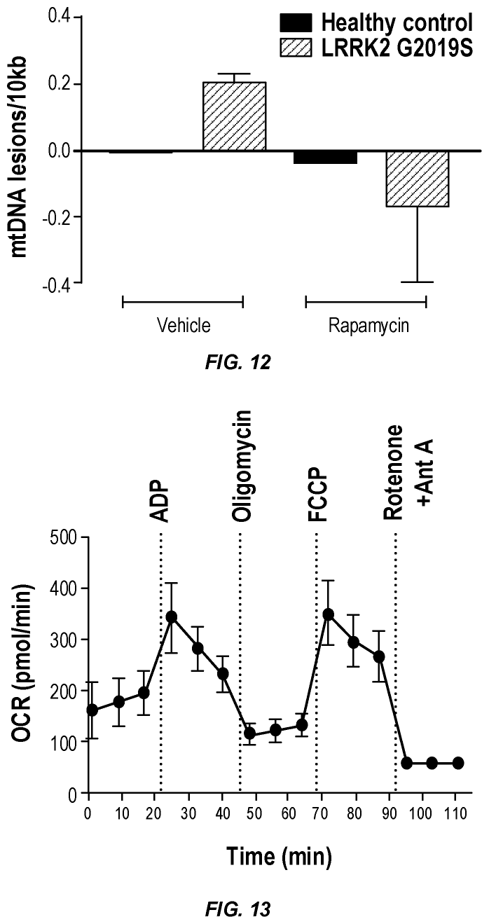

Experimental Design 2B: LRRK2 G2019S has been shown to reduce the efficiency of parkin/PINK1-mediated mitophagy, though the contribution of mitophagy to the protection of mitochondrial genome integrity in PD remains to be fully investigated. We will determine whether stimulating mitophagy may be a way to selectively purge mitochondria that have damaged genomes. Feasibility studies in LRRK2 G2019S patient iPSC-derived DA neurons showed that exposure to the autophagy inducer rapamycin restored mtDNA integrity (FIG. 12).

Using both pharmacological and genetic approaches, we will test whether blocking or stimulating mitophagy alters mtDNA damage levels. Mitophagy will be measured using the mitochondria-targeted mito-Keima, which is a pH sensitive fluorescent reporter resistant to lysosomal proteases and can be represented as a ratiometric heatmap. To block mitophagy, cells will be either exposed to the autophagosome inhibitor 3-methyladenine, vehicle control, or shRNA delivery to decrease a key protein in autophagy, autophagy related 5 (ATG5). To stimulate mitophagy, cells will be either exposed to rapamycin, vehicle control, or shRNA delivery to partially decrease levels of mitochondrial transport regulator Miro, a LRRK2-associated precursor event to mitophagy.

Experimental Design 2C: We hypothesize that mtDNA damage is due (at least in part), to mtDNA repair defects. The goal of Aim 2C is to define the mtDNA BER defects associated with LRRK2 mutations and idiopathic PD. We plan a comprehensive approach to determining the basal mitochondrial BER capacities (as described for the data in FIGS. 4A-4C). Mitochondrial extracts will be prepared in a suitable buffer. Data collected from both biochips will provide extensive functional information regarding the early steps of mitochondrial BER (and other DNA repair pathways). Based on these results, BER proteins will be examined to verify whether, in addition to activity, localization and/or protein levels are altered. Oligonucleotide-based assays will be performed, which allow for the specific probing of each enzymatic step individually. To determine if mtDNA repair defects are specific to BER or include additional DNA repair pathway defects, mtDNA repair capacity in response to different classes of DNA damaging agents will be explored. PD patient-derived DA neurons and LRRK2 mutant KI cells will be incubated with a wide range of concentrations with different classes of DNA damaging agents, such as a single-strand break inducer (i.e. methyl methanesulfonate), UV light, ionizing radiation, followed by a recovery period, to allow for mtDNA repair to occur. mtDNA lesions and copy number will be assessed as a function of concentration and time and mtDNA repair rates calculated. Cell viability will be assessed in parallel, as there may be inherent vulnerabilities to certain types of DNA damage due to DNA repair defects. Interestingly, ATM can downregulate BER in response to persistent DNA damage. ATM kinase activity will be inhibited and effects on mtDNA repair capacity determined.

The data generated will provide a complete understanding of the cellular pathways that drive mtDNA damage, by identifying the molecular mechanisms that preclude the repair of the mtDNA lesions. We will further delineate whether the mechanisms are shared or are distinct between LRRK2 mutations and idiopathic PD. Using a multidisciplinary approach and analysis centered on pathways related to mitochondrial homeostasis, these studies will yield detailed insights into the role of LRRK2 in mitochondrial function. This work will also establish an important link between mitochondrial pathways typically studied in isolation, with the mtDNA damage phenotype as the nexus. Decreased mtDNA repair capacity could also be related to a loss of imported mitochondrial DNA repair proteins; consistent with a report of an import defect in PD. Based on preliminary data, we predict early steps of BER activity in the mitochondria are decreased and a viral strategy to mitochondrially-target DNA repair proteins to restore mitochondrial genome integrity will help define precisely which step in the mtDNA repair pathway is impaired. In addition to mtDNA repair effects, ATM may directly impact Rabs and/or mitophagy and experiments to elucidate this possibility will be performed. Mutations in VPS35 (a subunit of the retromer cargo selective complex) enhance LRRK2-mediated Rab phosphorylation and wild-type VPS35 can regulate mitochondrial dynamics, suggesting a connection between endocytic trafficking complex and mitochondrial function and this alternative pathway may be investigated for its role in mitochondrial genome integrity. Due to the nature of the complex regulation of mitochondria and based on our preliminary data, future experiments may be needed to untangle the dependence and sequential relationship of relevant pathways contributing to mitochondrial dysfunction.

Aim 3: To Determine the Contribution of ATM Function to PD-Associated Phenotypes

Rationale: Perhaps, at first, a role for ATM in PD appears counterintuitive. Loss of the ATM protein is known to cause neurodegenerative disorders such as ataxia-telangiectasia. However, we have not observed a loss of ATM protein, but rather have distinct findings that suggest persistent activation of ATM in PD. Our preliminary data demonstrate that ATM is activated in both LRRK2 G2019S and environmentally-induced PD models (FIGS. 5A-5G). Downstream substrates of ATM, including γ-H2AX, pChk2 and p53 are up-regulated, further substantiating that ATM and the DNA damage response pathway are activated (FIG. 6, FIGS. 7A-7B). We have preliminary evidence that ATM is a substrate of LRRK2 (FIGS. 8A-8B). Furthermore, blocking ATM kinase activity rescued LRRK2 G2019S-induced mtDNA damage, suggesting the effects of ATM activity are downstream of LRRK2 activity (FIGS. 8A-8B). Independent literature supports our preliminary data and the premise that ATM is an important mediator of toxicity in PD. A quantitative phosphoproteomics screen by Dario Alessi's group and others identified activated ATM in LRRK2 G2019S but not wild-type mouse embryonic fibroblasts. In two different synucleinopathy mouse PD models, γ-H2AX foci are increased and ATM is activated in dopaminergic neurons. Expression of ATM pSer1981 was increased following treatment with the neurotoxin MPP+ and was increased in human postmortem brains. An overexpression system revealed a LRRK2-ATM interaction. Importantly, a functional relationship between parkin (another PD-linked gene) and ATM through mitochondria was recently reported. In a healthy cell, in order to cope with endogenous DNA damage, activation of ATM will initiate the DNA damage response to induce DNA repair. Activation of ATM must be tightly regulated (i.e. switched on/off as needed), in order to prevent toxic DNA repair, cell-cycle arrest, senescence or apoptosis. So in PD, persistent increased ATM kinase activity (akin to increased LRRK2 kinase activity) is likely a toxic gain of function. The contribution of ATM to mitochondrial function and pathology in PD has not been explored. Targeting ATM's kinase ameliorates toxicity and is neuroprotective in multiple models, including Huntington's disease. Therefore, the overall goal of Aim 3 is to define activation of the DNA damage response pathway and the outcomes of targeting ATM function. To do this, we will utilize multiple well-established in vitro cellular and in vivo PD models.

Experimental Design Aim 3A: ATM is localized predominantly in the nucleus, yet in some cells, there is a cytoplasmic ATM pool. Activated ATM on the other hand, even in the presence of DNA damaging agents, shows exclusively nuclear localization. Our preliminary data show an increase in predominantly nuclear expression with some aberrant cytoplasmic distribution of ATM pSer1981 in LRRK2^{G2019S/G2019S} KI HEK293 cells compared to isogenic controls (See U.S. Provisional Application No. 63/076,505, FIG. 16).

However, to our surprise, as discussed above, activated ATM did not appear to translocate to the mitochondria from the cytosol during LRRK2 activation, suggesting that this translocation to mitochondria is not necessary for LRRK2 G2019S-induced mtDNA damage. Our hypothesis is that sustained activation of ATM in LRRK2 mutant cells is pathological. The goal is to determine the abundance and subcellular localization of ATM. We will use the following models: (1) an endogenous model—LRRK2^{G2019S/G2019S} or LRRK2^{R1441C/R1441C} knock-in HEK293 cells compared to isogenic controls, and (2) LRRK2 mutant patient-derived and at-risk individuals carrying LRRK2 mutations or idiopathic PD iPSC-derived DA neurons (see Table 1 for full list of iPS-cell lines). To evaluate the abundance of ATM, cell lysates will be subjected to standard SDS-PAGE, followed by immunoblotting using antibodies against total and activated ATM. ATM mRNA expression will be quantified by RT-qPCR. To evaluate localization, subcellular fractionations using a standard sequential centrifugation protocol will be performed and determine if significant quantities of nuclear, cytoplasmic and mitochondrial ATM and ATM 1981 are present. As a corollary, immunocytochemistry using organelle-specific markers will be utilized on fixed cells to determine which cellular structures bind to ATM and ATM pSer1981, to investigate whether a portion of ATM associates with mitochondrial and/or other structures. Downstream targets and effectors such as γ-H2AX, pChk2 and p53 will be measured. We will also determine the time course and concentration-dependence of specific ATM kinase inhibitors (such as KU60019) that reverse the mtDNA phenotype. Target engagement will be verified by measuring ATM p1981. We will further test the dependency of ATM on the rapid and dynamic nature of the mtDNA damage phenotype. Cells will be treated with an ATM kinase inhibitor for about 2 hours, followed by a washout into media without inhibitor, to determine if the mtDNA damage phenotype is fully reestablished.

Experimental Design Aim 3B: We verified that mtDNA damage is increased specifically in the ventral midbrain in LRRK2 GKI mice compared to wild-type littermates (FIGS. 1A-1D) and that ATM is activated (FIG. 5), making this a suitable in vivo model to examine the effect of targeting ATM. To investigate the role of ATM in later classic PD pathology, rats will be treated with rotenone, a model which induces PD-like neurodegeneration. In rats, mtDNA damage is increased in the ventral midbrain after rotenone treatment; in vitro exposure activates ATM (FIGS. 5A-5G). The first objective of Aim 3B is to optimize the dosing paradigm of the newly-described brain-penetrant ATM kinase inhibitor AZD1390 and silence or overexpress ATM. In general, most drugs are not able to penetrate the blood-brain barrier, including the tool compound KU60019 that we used for our in vitro studies. However, a clinical-grade, potent and highly selective brain-penetrant ATM kinase inhibitor (AZD1390) was recently developed. In collaboration with AstraZeneca, we have access to this compound for our studies (personal communication with Dr. Stephen Durant). We will optimize both the dose and length of exposure to AZD1390.

Based on pharmacokinetics and target engagement performed in mice, a pilot experiment with wild-type, heterozygous and homozygous GKI mice and rotenone and vehicle exposed rats will be treated with two different doses of AZD1390 (2 mg/kg and 5 mg/kg) or vehicle for two and four weeks. Animals will be monitored for weight changes and overt toxicity. Dose and duration will be adjusted as necessary. Midbrain tissue will be collected and processed for Western blot and immunohistochemistry (IHC) to assess the effects of AZD1390 on target engagement of ATM pSer1981 in tyrosine hydroxylase (TH) neurons. We will optimize the dose and duration of treatment that decreases activated ATM levels to be comparable to wild-type, without affecting total ATM levels. Additionally, ATM will either be silenced or overexpressed, using shRNAs directed against ATM or a scrambled shRNA that doesn't correspond to any mouse/rat gene (control) or LV-mediated approach (LV-GFP-ATM or LV-GFP). Control and ATM targeted vectors will be stereotactically injected at equal titers into the SNc. Data show that the in vivo infection and gene transduction of ATM targeted vectors was successful. The hemisphere with control vector will serve as the control for the hemisphere that was injected with ATM vectors. Pilot studies will determine whether shRNA or LV-mediated targeting ATM, fully knocks down or increases this protein in vivo using IHC. Overall we will determine whether we can dose animals either prior or post mtDNA damage or ATM activation is detectable, to ascertain whether PD pathology or neurodegeneration can be prevented or reversed.

Rigor, Reproducibility, and Data Transparency:

Based on preliminary data for effect size and variability, we estimate that six animals for each condition would be necessary to detect a 20% difference with 80% power to reach α=0.05. All animal experiments will be performed with both male and female mice/rats. We will test for the significance of sex in the statistical models. If we observe a significant effect of sex in our measures, we will power our studies to consider sex as a biological factor by stratification. Litters will be randomized and experiments performed blinded to both genotype and treatment. The hypothesis that ATM function is important in protecting against LRRK2 or PD pathophysiological phenotypes will be tested. To do this, based on the optimized conditions for administration of AZD1390 or ATM-variants, (1) heterozygote and homozygote GKI mice and wild-type littermates or (2) rotenone relative to vehicle-treated rats will be exposed to either vehicle or AZD1390, or receive stereotaxic bilateral nigral injections of control vectors or viral-mediated silencing/overexpression of ATM and the following endpoints assessed: a) Mitochondrial dysfunction: Total and specific kinds of mtDNA damage by the PCR-based assay will be measured in the ventral midbrain and other relevant brain regions. Mitochondrial mutations will be measured using ultra-high accuracy Duplex Sequencing. Various other parameters of mitochondrial function will be investigated, including oxygen consumption rate (OCR), which will be measured using the Seahorse Bioanalyzer, as we have done previously; see feasibility data in (FIG. 18).

We will employ the methods Dr. Melrose's group describes for determining alterations in mitochondrial morphology, markers of dynamics, and OXPHOS assembly. Neuropathology and nigrostriatal neurodegeneration: It is important to acknowledge that one of the limitations of the GKI mice is the lack of certain markers associated with PD pathology, such as α-synuclein accumulation. Nonetheless, prior work has established an increase in tau phosphorylation in GKI mice, also a pathological hallmark associated with PD. Sections from substania nigra, striatum, and cortex will be probed in IHC for changes in total tau levels and tau phosphorylation (phospho-specific tau antibody at positions S202, T205) and alpha-syn 129 and formic acid alpha-syn in TH positive neurons. Total ATM and LRRK2 levels, and phosphorylation forms (e.g. ATM pSer1981, LRRK2 pSer1292) will be quantified. Total Rab10 and p-Rab10 will be quantified, in addition to other Rabs found to modulate mtDNA damage identified in Aim 2. For nigrostriatal neurodegeneration, IHC for the dopaminergic cell marker TH will be performed on sections from both the striatum and SN. If there is loss of dopaminergic terminals, a lesion will be evident in the striatum and will be quantitatively assessed as previously detailed by our lab. Overall Dr. Andrew West's (co-I) experience with LRRK2 transgenic and murine models will be invaluable in the execution of Aim 3 and we, therefore, do not anticipate technical hurdles that cannot be overcome.

We predict that therapeutically targeting ATM function will alleviate mitochondrial dysfunction in GKI and rotenone PD models. Whether restoring mitochondrial function is sufficient to prevent other PD-related pathology and neurodegeneration is unknown and is important to test. To further understand the role of ATM in LRRK2 pathogenesis, GKI mice may be crossed to ATM-deficient mice or conditional ATM point mutations that are catalytically inactive, or ATM that is mitochondrial or nuclear targeted. Alternatively, the ATR-mediated DNA damage response pathway may also be up-regulated, and pATR, pChk1 and downstream effectors will be measured to explore this possibility. If blocking LRRK2 and ATM kinase activity both rescue mtDNA damage, this suggests these two proteins are in the same pathway and has implications for LRRK2 therapeutics currently being tested in the clinic. Overall these experiments will allow us to determine whether ATM is a viable therapeutic target to pursue in PD.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Illustrative Sequences

SEQ ID NO: 1 (human LRRK2 amino acid sequence, wild type)
MASGSCQGCEEDEETLKKLIVRLNNVQEGKQIETLVQILEDLLVFTYSERASKLFQGKNI
HVPLLIVLDSYMRVASVQQVGWSLLCKLIEVCPGTMQSLMGPQDVGNDWEVLGVHQLILK
MLTVHNASVNLSVIGLKTLDLLLTSGKITLLILDEESDIFMLIFDAMHSFPANDEVQKLG
CKALHVLFERVSEEQLTEFVENKDYMILLSALTNFKDEEEIVLHVLHCLHSLAIPCNNVE
VLMSGNVRCYNIVVEAMKAFPMSERIQEVSCCLLHRLTLGNFFNILVLNEVHEFVVKAVQ
QYPENAALQISALSCLALLTETIFLNQDLEEKNENQENDDEGEEDKLFWLEACYKALTWH
RKNKHVQEAACWALNNLLMYQNSLHEKIGDEDGHFPAHREVMLSMLMHSSSKEVFQASAN
ALSTLLEQNVNFRKILLSKGIHLNVLELMQKHIHSPEVAESGCKMLNHLFEGSNTSLDIM
AAVVPKILTVMKRHETSLPVQLEALRAILHFIVPGMPEESREDTEFHHKLNMVKKQCFKN
EIQCLGLSLIGYLITKKNVFIGTGHLLAKILVSSLYRFKDVAEIQTKGFQTILAILKLSA
SFSKLLVHHSFDLVIFHQMSSNIMEQKDQQFLNLCCKCFAKVAMDDYLKNVMLERACDQN
NSIMVECLLLLGADANQAKEGSSLICQVCEKESSPKLVELLLNSGSREQDVRKALTISIG
KGDSQIISLLLRRLALDVANNSICLGGFCIGKVEPSWLGPLFPDKTSNLRKQTNIASTLA
RMVIRYQMKSAVEEGTASGSDGNFSEDVLSKFDEWTFIPDSSMDSVFAQSDDLDSEGSEG
SFLVKKKSNSISVGEFYRDAVLQRCSPNLQRHSNSLGPIFDHEDLLKRKRKILSSDDSLR
SSKLQSHMRHSDSISSLASEREYITSLDLSANELRDIDALSQKCCISVHLEHLEKLELHQ
NALTSFPQQLCETLKSLTHLDLHSNKFTSFPSYLLKMSCIANLDVSRNDIGPSVVLDPTV
ISSLSENFLEACPKVESFSARMNFLAAMPFLPPSMTILKLSQNKFSCIPEAILNLPHLRS
LDMSSNDIQYLPGPAHWKSLNLRELLFSHNQISILDLSEKAYLWSRVEKLHLSHNKLKEI
PPEIGCLENLTSLDVSYNLELRSFPNEMGKLSKIWDLPLDELHLNFDFKHIGCKAKDIIR
FLQQRLKKAVPYNRMKLMIVGNTGSGKTTLLQQLMKTKKSDLGMQSATVGIDVKDWPIQI
RDKRKRDLVLNVWDFAGREEFYSTHPHFMTQRALYLAVYDLSKGQAEVDAMKPWLFNIKA
RASSSPVILVGTHLDVSDEKQRKACMSKITKELLNKRGFPAIRDYHFVNATEESDALAKL
RKTIINESLNFKIRDQLVVGQLIPDCYVELEKIILSERKNVPIEFPVIDRKRLLQLVREN
QLQLDENELPHAVHFLNESGVLLHFQDPALQLSDLYFVEPKWLCKIMAQILTVKVEGCPK
HPKGIISRRDVEKFLSKKRKFPKNYMSQYFKLLEKFQIALPIGEEYLLVPSSLSDHRPVI
ELPHCENSEIIIRLYEMPYFPMGFWSRLINRLLEISPYMLSGRERALRPNRMYWRQGIYL
NWSPEAYCLVGSEVLDNHPESFLKITVPSCRKGCILLGQVVDHIDSLMEEWFPGLLEIDI
CGEGETLLKKWALYSFNDGEEHQKILLDDLMKKAEEGDLLVNPDQPRLTIPISQIAPDLI
LADLPRNIMLNNDELEFEQAPEFLLGDGSFGSVYRAAYEGEEVAVKIFNKHTSLRLLRQE
LVVLCHLHHPSLISLLAAGIRPRMLVMELASKGSLDRLLQQDKASLTRTLQHRIALHVAD
GLRYLHSAMIIYRDLKPHNVLLFTLYPNAAIIAKIADYGIAQYCCRMGIKTSEGTPGFRA
PEVARGNVIYNQQADVYSFGLLLYDILTTGGRIVEGLKFPNEFDELEIQGKLPDPVKEYG
CAPWPMVEKLIKQCLKENPQERPTSAQVFDILNSAELVCLTRRILLPKNVIVECMVATHH
NSRNASIWLGCGHTDRGQLSFLDLNTEGYTSEEVADSRILCLALVHLPVEKESWIVSGTQ
SGTLLVINTEDGKKRHTLEKMTDSVTCLYCNSFSKQSKQKNFLLVGTADGKLAIFEDKTV
KLKGAAPLKILNIGNVSTPLMCLSESTNSTERNVMWGGCGTKIFSFSNDFTIQKLIETRT
SQLFSYAAFSDSNIITVVVDTALYIAKQNSPVVEVWDKKTEKLCGLIDCVHFLREVMVKE
NKESKHKMSYSGRVKTLCLQKNTALWIGTGGGHILLLDLSTRRLIRVIYNFCNSVRVMMT
AQLGSLKNVMLVLGYNRKNTEGTQKQKEIQSCLTVWDINLPHEVQNLEKHIEVRKELAEK
MRRTSVE
SEQ ID NO: 2 (coding sequence for human LRRK2 protein, wild type)
ATGGCTAGTGGCAGCTGTCAGGGGTGCGAAGAGGACGAGGAAACTCTGAA
GAAGTTGATAGTCAGGCTGAACAATGTCCAGGAAGGAAAACAGATAGAAA
CGCTGGTCCAAATCCTGGAGGATCTGCTGGTGTTCACGTACTCCGAGCGC
GCCTCCAAGTTATTTCAAGGCAAAAATATCCATGTGCCTCTGTTGATCGT
CTTGGACTCCTATATGAGAGTCGCGAGTGTGCAGCAGGTGGGTTGGTCAC
TTCTGTGCAAATTAATAGAAGTCTGTCCAGGTACAATGCAAAGCTTAATG
GGACCCCAGGATGTTGGAAATGATTGGGAAGTCCTTGGTGTTCACCAATT
GATTCTTAAAATGCTAACAGTTCATAATGCCAGTGTAAACTTGTCAGTGA
TTGGACTGAAGACCTTAGATCTCCTCCTAACTTCAGGTAAAATCACCTTG
CTGATATTGGATGAAGAAAGTGATATTTTCATGTTAATTTTTGATGCCAT
GCACTCATTTCCAGCCAATGATGAAGTCCAGAAACTTGGATGCAAAGCTT
TACATGTGCTGTTTGAGAGAGTCTCAGAGGAGCAACTGACTGAATTTGTT
GAGAACAAAGATTATATGATATTGTTAAGTGCGTTAACAAATTTTAAAGA
TGAAGAGGAAATTGTGCTTCATGTGCTGCATTGTTTACATTCCCTAGCGA
TTCCTTGCAATAATGTGGAAGTCCTCATGAGTGGCAATGTCAGGTGTTAT
AATATTGTGGTGGAAGCTATGAAAGCATTCCCTATGAGTGAAAGAATTCA
AGAAGTGAGTTGCTGTTTGCTCCATAGGCTTACATTAGGTAATTTTTTCA
ATATCCTGGTATTAAACGAAGTCCATGAGTTTGTGGTGAAAGCTGTGCAG
CAGTACCCAGAGAATGCAGCATTGCAGATCTCAGCGCTCAGCTGTTTGGC
CCTCCTCACTGAGACTATTTTCTTAAATCAAGATTTAGAGGAAAAGAATG
AGAATCAAGAGAATGATGATGAGGGGGAAGAAGATAAATTGTTTTGGCTG
GAAGCCTGTTACAAAGCATTAACGTGGCATAGAAAGAACAAGCACGTGCA
GGAGGCCGCATGCTGGGCACTAAATAATCTCCTTATGTACCAAAACAGTT
TACATGAGAAGATTGGAGATGAAGATGGCCATTTCCCAGCTCATAGGGAA
GTGATGCTCTCCATGCTGATGCATTCTTCATCAAAGGAAGTTTTCCAGGC
ATCTGCGAATGCATTGTCAACTCTCTTAGAACAAAATGTTAATTTCAGAA
AAATACTGTTATCAAAAGGAATACACCTGAATGTTTTGGAGTTAATGCAG
AAGCATATACATTCTCCTGAAGTGGCTGAAAGTGGCTGTAAAATGCTAAA
TCATCTTTTTGAAGGAAGCAACACTTCCCTGGATATAATGGCAGCAGTGG
TCCCCAAAATACTAACAGTTATGAAACGTCATGAGACATCATTACCAGTG
CAGCTGGAGGCGCTTCGAGCTATTTTACATTTTATAGTGCCTGGCATGCC
AGAAGAATCCAGGGAGGATACAGAATTTCATCATAAGCTAAATATGGTTA
AAAAACAGTGTTTCAAGAATGATATTCACAAACTGGTCCTAGCAGCTTTG
AACAGGTTCATTGGAAATCCTGGGATTCAGAAATGTGGATTAAAAGTAAT
TTCTTCTATTGTACATTTTCCTGATGCATTAGAGATGTTATCCCTGGAAG
GTGCTATGGATTCAGTGCTTCACACACTGCAGATGTATCCAGATGACCAA
GAAATTCAGTGTCTGGGTTTAAGTCTTATAGGATACTTGATTACAAAGAA
GAATGTGTTCATAGGAACTGGACATCTGCTGGCAAAAATTCTGGTTTCCA
GCTTATACCGATTTAAGGATGTTGCTGAAATACAGACTAAAGGATTTCAG
ACAATCTTAGCAATCCTCAAATTGTCAGCATCTTTTTCTAAGCTGCTGGT
GCATCATTCATTTGACTTAGTAATATTCCATCAAATGTCTTCCAATATCA
TGGAACAAAAGGATCAACAGTTTCTAAACCTCTGTTGCAAGTGTTTTGCA
AAAGTAGCTATGGATGATTACTTAAAAAATGTGATGCTAGAGAGAGCGTG
TGATCAGAATAACAGCATCATGGTTGAATGCTTGCTTCTATTGGGAGCAG
ATGCCAATCAAGCAAAGGAGGGATCTTCTTTAATTTGTCAGGTATGTGAG
AAAGAGAGCAGTCCCAAATTGGTGGAACTCTTACTGAATAGTGGATCTCG
TGAACAAGATGTACGAAAAGCGTTGACGATAAGCATTGGGAAAGGTGACA
GCCAGATCATCAGCTTGCTCTTAAGGAGGCTGGCCCTGGATGTGGCCAAC
AATAGCATTTGCCTTGGAGGATTTTGTATAGGAAAAGTTGAACCTTCTTG
GCTTGGTCCTTTATTTCCAGATAAGACTTCTAATTTAAGGAAACAAACAA
ATATAGCATCTACACTAGCAAGAATGGTGATCAGATATCAGATGAAAAGT
GCTGTGGAAGAAGGAACAGCCTCAGGCAGCGATGGAAATTTTTCTGAAGA
TGTGCTGTCTAAATTTGATGAATGGACCTTTATTCCTGACTCTTCTATGG
ACAGTGTGTTTGCTCAAAGTGATGACCTGGATAGTGAAGGAAGTGAAGGC
TCATTTCTTGTGAAAAAGAAATCTAATTCAATTAGTGTAGGAGAATTTTA
CCGAGATGCCGTATTACAGCGTTGCTCACCAAATTTGCAAAGACATTCCA
ATTCCTTGGGGCCCATTTTTGATCATGAAGATTTACTGAAGCGAAAAAGA
AAAATATTATCTTCAGATGATTCACTCAGGTCATCAAAACTTCAATCCCA
TATGAGGCATTCAGACAGCATTTCTTCTCTGGCTTCTGAGAGAGAATATA
TTACATCACTAGACCTTTCAGCAAATGAACTAAGAGATATTGATGCCCTA
AGCCAGAAATGCTGTATAAGTGTTCATTTGGAGCATCTTGAAAAGCTGGA
GCTTCACCAGAATGCACTCACGAGCTTTCCACAACAGCTATGTGAAACTC
TGAAGAGTTTGACACATTTGGACTTGCACAGTAATAAATTTACATCATTT
CCTTCTTATTTGTTGAAAATGAGTTGTATTGCTAATCTTGATGTCTCTCG
AAATGACATTGGACCCTCAGTGGTTTTAGATCCTACAGTGAAATGTCCAA
CTCTGAAACAGTTTAACCTGTCATATAACCAGCTGTCTTTTGTACCTGAG
AACCTCACTGATGTGGTAGAGAAACTGGAGCAGCTCATTTTAGAAGGAAA
TAAAATATCAGGGATATGCTCCCCCTTGAGACTGAAGGAACTGAAGATTT
TAAACCTTAGTAAGAACCACATTTCATCCCTATCAGAGAACTTTCTTGAG
GCTTGTCCTAAAGTGGAGAGTTTCAGTGCCAGAATGAATTTTCTTGCTGC
TATGCCTTTCTTGCCTCCTTCTATGACAATCCTAAAATTATCTCAGAACA
AATTTTCCTGTATTCCAGAAGCAATTTTAAATCTTCCACACTTGCGGTCT
TTAGATATGAGCAGCAATGATATTCAGTACCTACCAGGTCCCGCACACTG
GAAATCTTTGAACTTAAGGGAACTCTTATTTAGCCATAATCAGATCAGCA
TCTTGGACTTGAGTGAAAAAGCATATTTATGGTCTAGAGTAGAGAAACTG
CATCTTTCTCACAATAAACTGAAAGAGATTCCTCCTGAGATTGGCTGTCT
TGAAAATCTGACATCTCTGGATGTCAGTTACAACTTGGAACTAAGATCCT
TTCCCAATGAAATGGGGAAATTAAGCAAAATATGGGATCTTCCTTTGGAT
GAACTGCATCTTAACTTTGATTTTAAACATATAGGATGTAAAGCCAAAGA
CATCATAAGGTTTCTTCAACAGCGATTAAAAAAGGCTGTGCCTTATAACC
GAATGAAACTTATGATTGTGGGAAATACTGGGAGTGGTAAAACCACCTTA
TTGCAGCAATTAATGAAAACCAAGAAATCAGATCTTGGAATGCAAAGTGC
CACAGTTGGCATAGATGTGAAAGACTGGCCTATCCAAATAAGAGACAAAA
GAAAGAGAGATCTCGTCCTAAATGTGTGGGATTTTGCAGGTCGTGAGGAA
TTCTATAGTACTCATCCCCATTTTATGACGCAGCGAGCATTGTACCTTGC
TGTCTATGACCTCAGCAAGGGACAGGCTGAAGTTGATGCCATGAAGCCTT
GGCTCTTCAATATAAAGGCTCGCGCTTCTTCTTCCCCTGTGATTCTCGTT
GGCACACATTTGGATGTTTCTGATGAGAAGCAACGCAAAGCCTGCATGAG
TAAAATCACCAAGGAACTCCTGAATAAGCGAGGGTTCCCTGCCATACGAG
ATTACCACTTTGTGAATGCCACCGAGGAATCTGATGCTTTGGCAAAACTT
CGGAAAACCATCATAAACGAGAGCCTTAATTTCAAGATCCGAGATCAGCT
TGTTGTTGGACAGCTGATTCCAGACTGCTATGTAGAACTTGAAAAAATCA
TTTTATCGGAGCGTAAAAATGTGCCAATTGAATTTCCCGTAATTGACCGG
AAACGATTATTACAACTAGTGAGAGAAAATCAGCTGCAGTTAGATGAAAA
TGAGCTTCCTCACGCAGTTCACTTTCTAAATGAATCAGGAGTCCTTCTTC
ATTTTCAAGACCCAGCACTGCAGTTAAGTGACTTGTACTTTGTGGAACCC
AAGTGGCTTTGTAAAATCATGGCACAGATTTTGACAGTGAAAGTGGAAGG
TTGTCCAAAACACCCTAAGGGCATTATTTCGCGTAGAGATGTGGAAAAAT
TTCTTTCAAAAAAAAGGAAATTTCCAAAGAACTACATGTCACAGTATTTT
AAGCTCCTAGAAAAATTCCAGATTGCTTTGCCAATAGGAGAAGAATATTT
GCTGGTTCCAAGCAGTTTGTCTGACCACAGGCCTGTGATAGAGCTTCCCC
ATTGTGAGAACTCTGAAATTATCATCCGACTATATGAAATGCCTTATTTT
CCAATGGGATTTTGGTCAAGATTAATCAATCGATTACTTGAGATTTCACC
TTACATGCTTTCAGGGAGAGAACGAGCACTTCGCCCAAACAGAATGTATT
GGCGACAAGGCATTTACTTAAATTGGTCTCCTGAAGCTTATTGTCTGGTA
GGATCTGAAGTCTTAGACAATCATCCAGAGAGTTTCTTAAAAATTACAGT
TCCTTCTTGTAGAAAAGGCTGTATTCTTTTGGGCCAAGTTGTGGACCACA
TTGATTCTCTCATGGAAGAATGGTTTCCTGGGTTGCTGGAGATTGATATT
TGTGGTGAAGGAGAAACTCTGTTGAAGAAATGGGCATTATATAGTTTTAA
TGATGGTGAAGAACATCAAAAAATCTTACTTGATGACTTGATGAAGAAAG
CAGAGGAAGGAGATCTCTTAGTAAATCCAGATCAACCAAGGCTCACCATT
CCAATATCTCAGATTGCCCCTGACTTGATTTTGGCTGACCTGCCTAGAAA
TATTATGTTGAATAATGATGAGTTGGAATTTGAACAAGCTCCAGAGTTTC
TCCTAGGTGATGGCAGTTTTGGATCAGTTTACCGAGCAGCCTATGAAGGA
GAAGAAGTGGCTGTGAAGATTTTTAATAAACATACATCACTCAGGCTGTT
AAGACAAGAGCTTGTGGTGCTTTGCCACCTCCACCACCCCAGTTTGATAT
CTTTGCTGGCAGCTGGGATTCGTCCCCGGATGTTGGTGATGGAGTTAGCC
TCCAAGGGTTCCTTGGATCGCCTGCTTCAGCAGGACAAAGCCAGCCTCAC
TAGAACCCTACAGCACAGGATTGCACTCCACGTAGCTGATGGTTTGAGAT
ACCTCCACTCAGCCATGATTATATACCGAGACCTGAAACCCCACAATGTG
CTGCTTTTCACACTGTATCCCAATGCTGCCATCATTGCAAAGATTGCTGA
CTACGGCATTGCTCAGTACTGCTGTAGAATGGGGATAAAAACATCAGAGG
GCACACCAGGGTTTCGTGCACCTGAAGTTGCCAGAGGAAATGTCATTTAT
AACCAACAGGCTGATGTTTATTCATTTGGTTTACTACTCTATGACATTTT
GACAACTGGAGGTAGAATAGTAGAGGGTTTGAAGTTTCCAAATGAGTTTG
ATGAATTAGAAATACAAGGAAAATTACCTGATCCAGTTAAAGAATATGGT
TGTGCCCCATGGCCTATGGTTGAGAAATTAATTAAACAGTGTTTGAAAGA
AAATCCTCAAGAAAGGCCTACTTCTGCCCAGGTCTTTGACATTTTGAATT
CAGCTGAATTAGTCTGTCTGACGAGACGCATTTTATTACCTAAAAACGTA
ATTGTTGAATGCATGGTTGCTACACATCACAACAGCAGGAATGCAAGCAT
TTGGCTGGGCTGTGGGCACACCGACAGAGGACAGCTCTCATTTCTTGACT
TAAATACTGAAGGATACACTTCTGAGGAAGTTGCTGATAGTAGAATATTG
TGCTTAGCCTTGGTGCATCTTCCTGTTGAAAAGGAAAGCTGGATTGTGTC
TGGGACACAGTCTGGTACTCTCCTGGTCATCAATACCGAAGATGGGAAAA
AGAGACATACCCTAGAAAAGATGACTGATTCTGTCACTTGTTTGTATTGC
AATTCCTTTTCCAAGCAAAGCAAACAAAAAAATTTTCTTTTGGTTGGAAC
CGCTGATGGCAAGTTAGCAATTTTTGAAGATAAGACTGTTAAGCTTAAAG
GAGCTGCTCCTTTGAAGATACTAAATATAGGAAATGTCAGTACTCCATTG
ATGTGTTTGAGTGAATCCACAAATTCAACGGAAAGAAATGTAATGTGGGG
AGGATGTGGCACAAAGATTTTCTCCTTTTCTAATGATTTCACCATTCAGA
AACTCATTGAGACAAGAACAAGCCAACTGTTTTCTTATGCAGCTTTCAGT
GATTCCAACATCATAACAGTGGTGGTAGACACTGCTCTCTATATTGCTAA
GCAAAATAGCCCTGTTGTGGAAGTGTGGGATAAGAAAACTGAAAAACTCT
GTGGACTAATAGACTGCGTGCACTTTTTAAGGGAGGTAATGGTAAAAGAA
AACAAGGAATCAAAACACAAAATGTCTTATTCTGGGAGAGTGAAAACCCT
CTGCCTTCAGAAGAACACTGCTCTTTGGATAGGAACTGGAGGAGGCCATA
TTTTACTCCTGGATCTTTCAACTCGTCGACTTATACGTGTAATTTACAAC
TTTTGTAATTCGGTCAGAGTCATGATGACAGCACAGCTAGGAAGCCTTAA
AAATGTCATGCTGGTATTGGGCTACAACCGGAAAAATACTGAAGGTACAC
AAAAGCAGAAAGAGATACAATCTTGCTTGACCGTTTGGGACATCAATCTT
CCACATGAAGTGCAAAATTTAGAAAAACACATTGAAGTGAGAAAAGAATT
AGCTGAAAAAATGAGACGAACATCTGTTGAGTAA
SEQ ID NO: 3 (human ATM protein, wild type)
MSLVLNDLLICCRQLEHDRATERKKEVEKFKRLIRDPETIKHLDRHSDSKQGKYLNWDAV
FRFLQKYIQKETECLRIAKPNVSASTQASRQKKMQEISSLVKYFIKCANRRAPRLKCQEL
LNYIMDTVKDSSNGAIYGADCSNILLKDILSVRKYWCEISQQQWLELFSVYFRLYLKPSQ
DVHRVLVARIIHAVTKGCCSQTDGLNSKFLDFFSKAIQCARQEKSSSGLNHILAALTIFL
KTLAVNFRIRVCELGDEILPTLLYIWTQHRLNDSLKEVIIELFQLQIYIHHPKGAKTQEK
GAYESTKWRSILYNLYDLLVNEISHIGSRGKYSSGFRNIAVKENLIELMADICHQVFNED
TRSLEISQSYTTTQRESSDYSVPCKRKKIELGWEVIKDHLQKSQNDFDLVPWLQIATQLI
LKLWNKIWCITFRGISSEQIQAENFGLLGAIIQGSLVEVDREFWKLFTGSACRPSCPAVC
CLTLALTTSIVPGTVKMGIEQNMCEVNRSFSLKESIMKWLLFYQLEGDLENSTEVPPILH
SNFPHLVLEKILVSLTMKNCKAAMNFFQSVPECEHHQKDKEELSFSEVEELFLQTTFDKM
DFLTIVRECGIEKHQSSIGFSVHQNLKESLDRCLLGLSEQLLNNYSSEITNSETLVRCSR
LLVGVLGCYCYMGVIAEEEAYKSELFQKAKSLMQCAGESITLFKNKTNEEFRIGSLRNMM
QLCTRCLSNCTKKSPNKIASGFFLRLLTSKLMNDIADICKSLASFIKKPFDRGEVESMED
DTNGNLMEVEDQSSMNLFNDYPDSSVSDANEPGESQSTIGAINPLAEEYLSKQDLLFLDM
LKFLCLCVTTAQTNTVSFRAADIRRKLLMLIDSSTLEPTKSLHLHMYLMLLKELPGEEYP
LPMEDVLELLKPLSNVCSLYRRDQDVCKTILNHVLHVVKNLGQSNMDSENTRDAQGQFLT
VIGAFWHLTKERKYIFSVRMALVNCLKTLLEADPYSKWAILNVMGKDFPVNEVFTQFLAD
NHHQVRMLAAESINRLFQDTKGDSSRLLKALPLKLQQTAFENAYLKAQEGMREMSHSAEN
PETLDEIYNRKSVLLTLIAVVLSCSPICEKQALFALCKSVKENGLEPHLVKKVLEKVSET
FGYRRLEDFMASHLDYLVLEWLNLQDTEYNLSSFPFILLNYTNIEDFYRSCYKVLIPHLV
IRSHFDEVKSIANQIQEDWKSLLTDCFPKILVNILPYFAYEGTRDSGMAQQRETATKVYD
MLKSENLLGKQIDHLFISNLPEIVVELLMTLHEPANSSASQSTDLCDFSGDLDPAPNPPH
FPSHVIKATFAYISNCHKTKLKSILEILSKSPDSYQKILLAICEQAAETNNVYKKHRILK
IYHLFVSLLLKDIKSGLGGAWAFVLRDVIYTLIHYINQRPSCIMDVSLRSFSLCCDLLSQ
VCQTAVTYCKDALENHLHVIVGTLIPLVYEQVEVQKQVLDLLKYLVIDNKDNENLYITIK
LLDPFPDHVVFKDLRITQQKIKYSRGPFSLLEEINHFLSVSVYDALPLTRLEGLKDLRRQ
IDFSTIAIQHSKDASYTKALKLFEDKELQWTFIMLTYLNNTLVEDCVKVRSAAVTCLKNI
LATKTGHSFWEIYKMTTDPMLAYLQPFRTSRKKFLEVPRFDKENPFEGLDDINLWIPLSE
NHDIWIKTLTCAFLDSGGTKCEILQLLKPMCEVKTDFCQTVLPYLIHDILLQDTNESWRN
LLSTHVQGFFTSCLRHFSQTSRSTTPANLDSESEHFFRCCLDKKSQRTMLAVVDYMRRQK
RPSSGTIFNDAFWLDLNYLEVAKVAQSCAAHFTALLYAEIYADKKSMDDQEKRSLAFEEG
SQSTTISSLSEKSKEETGISLQDLLLEIYRSIGEPDSLYGCGGGKMLQPITRLRTYEHEA
QAAWRNMQWDHCTSVSKEVEGTSYHESLYNALQSLRDREFSTFYESLKYARVKEVEEMCK
RSLESVYSLYPTLSRLQAIGELESIGELFSRSVTHRQLSEVYIKWQKHSQLLKDSDFSFQ
EPIMALRTVILEILMEKEMDNSQRECIKDILTKHLVELSILARTFKNTQLPERAIFQIKQ
YNSVSCGVSEWQLEEAQVFWAKKEQSLALSILKQMIKKLDASCAANNPSLKLTYTECLRV
CGNWLAETCLENPAVIMQTYLEKAVEVAGNYDGESSDELRNGKMKAFLSLARFSDTQYQR
IENYMKSSEFENKQALLKRAKEEVGLLREHKIQTNRYTVKVQRELELDELALRALKEDRK
RFLCKAVENYINCLLSGEEHDMWVFRLCSLWLENSGVSEVNGMMKRDGMKIPTYKFLPLM
YQLAARMGTKMMGGLGFHEVLNNLISRISMDHPHHTLFIILALANANRDEFLTKPEVARR
SRITKNVPKQSSQLDEDRTEAANRIICTIRSRRPQMVRSVEALCDAYIILANLDATQWKT
QRKGINIPADQPITKLKNLEDVVVPTMEIKVDHTGEYGNLVTIQSFKAEFRLAGGVNLPK
IIDCVGSDGKERRQLVKGRDDLRQDAVMQQVFQMCNTLLQRNTETRKRKLTICTYKVVPL
SQRSGVLEWCTGTVPIGEFLVNNEDGAHKRYRPNDFSAFQCQKKMMEVQKKSFEEKYEVF
MDVCQNFQPVFRYFCMEKFLDPAIWFEKRLAYTRSVATSSIVGYILGLGDRHVQNILINE
QSAELVHIDLGVAFEQGKILPTPETVPFRLTRDIVDGMGITGVEGVFRRCCEKTMEVMRN
SQETLLTIVEVLLYDPLFDWTMNPLKALYLQQRPEDETELHPTLNADDQECKRNLSDIDQ
SFNKVAERVLMRLQEKLKGVEEGTVLSVGGQVNLLIQQAIDPKNLSRLFPGWKAWV
SEQ ID NO: 4
5′-TCT AAG CCT CCT TATTCG AGC CGA-3′
SEQ ID NO: 5
5′-TTT CAT CAT GCG GAGATG TTG GAT GG-3′
SEQ ID NO: 6
5′-CAT GTC ACC ACT GGACTC TGC AC-3′
SEQ ID NO: 7
5′-CCT GGA GTA GGA ACA AAA ATT GCT G-3′
SEQ ID NO: 8
5′-CCC CAC AAA CCC CATTAC TAA ACC CA-3′
SEQ ID NO: 9
5′-TTT CAT CAT GCG GAGATG TTG GAT GG-3′

What is claimed is:

1. A method of treating a neurological disorder comprising administering to the subject a therapeutically effective amount of an ATM inhibitor.

2. The method of claim 1, wherein the subject expresses a LRRK2 variant protein, wherein the LRRK2 variant protein has increased kinase activity as compared to a wild type LRRK2 protein.

3. The method of claim 2, wherein the LRRK2 variant protein comprises one or more amino acid substitutions relative to SEQ ID NO: 1.

4. The method of claim 2, wherein the one or more amino acid substitutions comprise one or more of G2019S, R1441C, R1441G, R1441H, Y1699C, or I2020T.

5. The method of claim 2, wherein the LRRK2 variant protein has an amino acid sequence that is at least 90% identical to SEQ ID NO: 1 and comprises one or more of G2019S, R1441C, R1441G, R1441H, Y1699C, or I2020T.

6. The method of claim 2, wherein brain cells of the subject have a higher level of mitochondria DNA damage as compared to brain cells of a control subject.

7. The method of claim 6, wherein the mitochondria DNA damage is oxidized mtDNA lesions or presence of abasic sites in the mitochondria DNA.

8. The method of claim 6, wherein the brain cells are dopaminergic neurons of the subject.

9. The method of claim 6, wherein the brain cells are located in the ventral midbrain region.

10. The method of claim 1, wherein the neurological disorder comprises a neurodegenerative disease.

11. The method according to claim 10, wherein the neurodegenerative disease is any one of Alzheimer's disease, Mild Cognitive Impairment, Pick's disease, Parkinson's disease, Huntington's disease, or a prion-associated disease.

12. The method according to claim 11 in which the neurodegenerative disease comprises Parkinson's disease.

13. The method of claim 1 in which the ATM kinase inhibitor is any one of KU-55933, Dactolisib (BEZ235), KU-60019, JU-59403, AZ31, AZ32, AZD0156, AZD1390, VE-821, Wortmannin, Torin 2, CP-466722, Berzosertib (VE-822), and the like.

14. The method according to claim 13 in which the ATM kinase inhibitor comprises AZD1390.

15. The method according to claim 14 in which the ATM kinase inhibitor comprises KU60019.

16. A method of rescuing LRRK2 genome instability in a subject suffering from a neurological disorder, the method comprising administering to the subject a therapeutically effective amount of an agent that inhibits the activity of ATM such that LRRK2 genome stability is rescued.

17. The method of claim 16, wherein rescue of LRRK2 genome stability is indicated by a DNA lesion frequency in the subject that is substantially identical to the DNA lesion frequency in a control subject that expresses a wild type LRRK2 protein.

18. The method of claim 1, wherein the method further comprises administering one or more additional therapeutic agents.

19. A kit comprising:
(a) primers for detecting a genomic sequence encoding a LRRK2 variant protein in a biological sample, wherein the LRRK2 variant protein has increased kinase activity relative to wild type LRRK2, and
(b) instructions for identifying the genomic sequence as encoding the LRRK2 variant protein.

20. The kit of claim 19, wherein the LRRK2 variant protein has one or more of a G2019S mutation, an R1441C mutation, an R1441G mutation, an R1441H mutation, a Y1699C mutation, or an 12020T mutation.
